(19) United States
(12) Patent Application Publication
(10) Pub. No.: US 2002/0151487 A1
(43) Pub. Date: Oct. 17, 2002

(54) Method and reagents for epithelial barrier formation and treatment of malignant and benign skin disorders by modulating the notch pathway

(75) Inventors: Brian J. Nickoloff, Burr Ridge, IL (US); Lucio Miele, Wayne Township, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Loyola University Chicago, Maywood, IL

(21) Appl. No.: 09/944,849

(22) Filed: Aug. 31, 2001

Related U.S. Application Data
(60) Provisional application No. 60/229,614, filed on Aug. 31, 2000.

(51) Int. Cl. A61K 48/00; A61K 38/17; C12Q 1/68; C07H 21/04

(52) U.S. Cl. 514/12; 514/44; 536/23.2; 435/6

(57) ABSTRACT

In one embodiment, the invention provides a method of inducing differentiation of an epithelial cell. In another embodiment, the invention provides a method for inducing formation of a barrier within epithelium. In another embodiment, the invention provides a method for producing differentiated epidermis. In another embodiment, the invention provides a method of assaying for genetic propensity of a patient to develop a disorder associated with epithelial barrier formation. In anther embodiment, the invention provides a diagnostic test to determine the expression levels of Notch ligands, receptors, or other Notch signaling compounds in cells. In another embodiment, the invention provides novel Notch ligands. In another embodiment, the invention provides a method of preventing or retarding the progression of a benign or malignant disorder in skin.

### FIELD OF THE INVENTION

This invention pertains to methods and reagents for epithelial barrier formation and treatment of malignant and benign skin disorders.

### BACKGROUND OF THE INVENTION

Among their structural features, epithelial tissues (e.g., skin and mucosal) contain a typically nonviable barrier layer. Such layers provide various essential functions to an animal, among which are the retention of water, exclusion of hostile elements of the environment, such as toxins, allergens or pathogens (e.g., microbes such as bacteria, fungal spores, and viruses, other parasites, etc.).

The barrier of the skin (stratum corneum) is formed of non-viable anucleate keratinocytes that have undergone a differentiation and apoptotic events to become corneocytes. While the physical and chemical constituents of the stratum corneum have been well studied (see, e.g., Elias, J. Dermatol., 23, 756-68 (1996); Holleran et al., J. Clin. Invest., 91, 1656-1644 (1993)), the molecular events underlying the growth-arrest, terminal differentiation, and apoptosis of keratinocytes, and corneogenesis remain elusive (see, e.g., Roop, Science, 267, 474-75 (1995)). Currently, the most widely studied inducer of keratinocyte differentiation is the calcium ion. A calcium gradient is present in the epidermis, with a low concentration in the proliferative basal cell layer progressively increasing towards the surface (Menon et al., Cell. Tissue Res., 270, 503-12 (1992)). Moreover, cultured keratinocytes can be induced to proliferate in low calcium concentrations, or they can be induced to withdraw from the cell cycle upon exposure to high calcium concentration (Yuspa et al., J. Cell. Biol., 109, 1207-17 (1989)). Despite such observations, neither elevated calcium concentration nor other agents have been able to promote keratinocyte differentiation or stratification using human keratinocytes (see, e.g., Green, Cell, 11, 405-16 (1977)). Thus, there remains a need for a method and reagents for inducing terminal differentiation and apoptosis of keratinocytes thereby resulting in an orderly stratified and cornified epithelium.

Many disorders involving epithelial tissues are associated with dysfunctional epithelial barriers. For example, the loss of integrity of the stratum corneum in skin can have any of a wide variety of medical consequences, such as increased risk of infection, excessive water loss, dermal irritation (e.g., dryness or itching), and other dermatological problems. Examples of such disorders include actinic keratosis, aged skin, alopecia (e.g., androgenic alopecia, alopecia areata, etc.), asteototic skin (dry skin, winter itch), Bowen's disease, cancers (e.g., keratocanthoma, squamous cell carcinoma, basal cell carcinoma, etc.), dermatitis (e.g., atopic dermatitis, allergic/irritant contact dermatitis, etc.), drug reactions, ichthyotic skin, photodamaged skin, psoriasis, sunburn, incontinentia pigmenti, and the like. Similarly, other epithelial organ systems, such as oral mucosal, gastrointestinal tract, pulmonary system, etc., also are dependent on formation of an effective barrier to shield against noxious agents at the sites of interfacing between the host and environment. As such, many diseases of these extracutaneous sites also can result from abnormal barrier formation and/or function. Moreover, altered cellular differentiation (e.g., of keratinocytes), and particularly a block in terminal differentiation, can predispose an individual to the development of various skin cancers and other epithelial-derived neoplasms at extracutaneous sites (see, e.g., Yuspa et al, Cancer Res., Res., 54, 1178-89 (1994)).

In part due to the current incomplete understanding of the cellular and molecular events involved with proper barrier formation, current therapy for disorders involving epithelial barrier dysfunction and altered differentiation is sub-optimal. Typical treatments for such disorders include emollients, Retinoic Acid derivatives (e.g., vitamin A), corticosteroids, α-hydroxy acids, phototherapy, Vitamin D derivatives, 5-fluorouracil, cyclosporin A, methotrexate, etc. Even when they are effective, the clinical results of such treatments typically are non-specific. Moreover, many of these treatments produce unpleasant side effects in patients, such as dry skin, irritation, contact dermatitis, immunosuppression, atrophy, metabolic problems (e.g., hypercalcemia), mutagenesis (which can lead to cancer), and other unpleasant side effects. Thus, there remains a need for a method of treating disorders associated with dysfunctional epithelial barrier formation, within both cutaneous and extracutaneous tissues.

Also due in part to the incomplete understanding regarding the genetic and molecular biology of epithelial barrier formation, advanced diagnosis of many such disorders, or genetic propensities to contract such disorders, currently cannot be conducted. Thus, there remains a need for methods and reagents for diagnosing or determining susceptibility to disorders associated with dysfunctional epithelial barrier formation.

Among the deadliest of all skin diseases, and often most difficult to treat are skin cancers. The epidermis is a confederacy of three fundamental cell types: melanocytes, keratinocytes, and T-cells. Cell of each of these three types of cells can become cancerous: melanocytes giving rise to melanoma and keratinocytes giving rise to basal and squamous cell carcinoma, and T-cells giving rise to lymphoma. While each of these types of skin cancers is distinguishable, each progress from pre-malignancy through three stages of severity. In the first stage, each of these three types of cancer presents in situ within the epidermis as tumors or lesions that have not penetrated the basement membrane of the epidermis. Such skin cancers also sometimes are referred to as “pre-invasive” cancers. While skin cancers can exist for long periods of time as in situ cancers, many such cancers progress to a second, “early invasive” stage during which some cells penetrate the basement membrane of the epidermis and enter the dermis with access to blood and lymphatic vessels. Cancers of this stage are often diagnosed as being “low-grade,” and cells of the first two stages can be classified as “non-aggressive (a term particularly appropriate for T-cell lymphomas, some of which, while presenting in the skin, can have originated in other tissues). The final stage of skin cancer progression is an aggressive (often described as “advanced” or “high-grade”) stage, during which the cancer actively invades beneath the epidermis and into dermis and frequently metastasizes.

Treatment and prognoses of skin cancers can depend on their stage. For example, in situ and often non-aggressive melanoma typically can be readily excised by standard surgical techniques. As melanoma progresses, however, it spreads vertically through the layers of the skin, quickly metastasizing. Once melanoma enters its aggressive stage, it represents one of the most difficult of all cancers to treat, as it responds poorly to most chemotherapeutic and other anticancer treatments. As another example, cutaneous T-cell lymphoma (CTCL) is a fairly common form of non-Hodkins lymphoma that typically presents in skin. CTCL begins when T-cells become malignant and proliferate in situ within the epidermis to form patches, plaques, and tumor nodules. Such cells exhibit a propensity to proliferate within epidermis, and such lesions can be readily excised from patients. As CTCL progresses, however, its epidermotropism diminishes and is lost as the disease enters the aggressive phase, during which the malignant cells typically metastasizes and leads to leukemia. It is much more difficult to treat at this point.

The molecular bases for the behavior of skin cancers during the three stages, as well as the bases for their transition from one stage to the next is poorly understood. Because of the absence of molecular markers, diagnosis of both the type and phase of skin cancer typically is accomplished by light microscopical, histological, examination of biopsied tissue. Using such methods, it is well within the skill of the art to distinguish among the various types of skin cancers (e.g., melanoma, CTCL, basal cell carcinoma, squamous cell carcinoma, etc.). However, the absence of molecular markers renders it difficult at times to distinguish between the stage to which a given cancer has progressed, particularly distinguishing between the early-invasive and advanced stages. Thus, there continues to exist a need for additional methods for diagnosing and treating skin cancers.

### BRIEF SUMMARY OF THE INVENTION

In one embodiment, the invention provides a method of inducing differentiation of an epithelial cell. In accordance with the method, a source of a Notch agonist is supplied to the epithelial cell such that the Notch Pathway is activated within the epithelial cell to induce differentiation of the epithelial cell. In another embodiment, the invention provides a method for inducing formation of a barrier within epithelium. In accordance with this aspect of the invention, a source of a Notch agonist is delivered to cells within the epithelium such that they are induced to form a barrier within the epithelium. In another embodiment, the invention provides a method for producing differentiated epidermis. In accordance with this aspect of the invention, undifferentiated epidermal tissue is cultured in the presence of a source of a Notch agonist such that the Notch Pathway is activated within at least one cell of the epidermal tissue so as to induce differentiation of the epidermis. In another embodiment, the invention provides a method of retarding the progression of an epithelial malignancy. In accordance with this aspect, a source of a Notch agonist or antagonist is provided to at least one malignant or pre-malignant epithelial cell such that the Notch pathway is activated or attenuated in the cell to retard its progression towards malignancy.

In another embodiment, the invention provides a method of assaying for genetic propensity of a patient to develop a disorder associated with epithelial barrier formation. In accordance with this method, DNA or RNA is obtained from the patient, and a characteristic of the DNA or RNA from the patient encoding a Notch protein or a Notch ligand is deduced. Finally, the patient's characteristic is compared to the wild-type characteristic to determine whether the patient's DNA or RNA corresponds to the wild-type counterpart. In anther embodiment, the invention provides a diagnostic test to determine the expression levels of Notch ligands, receptors, or other Notch signaling compounds in cells. In another embodiment, the invention provides novel Notch ligands. Such reagents can be employed to activate the Notch pathway, such as for use in the methods of the present invention.

In another embodiment, the invention provides a method of diagnosing aggressive melanoma or CTCL by assaying for the expression pattern or overexpression of Notch receptors or ligands.

The methods and reagents of the present invention provide tools for further research, for example into epithelial growth, differentiation and regulation of apoptosis. In some embodiments, the methods and reagents can have clinical application, for example as therapeutic methods or to grow epithelial graft tissue (e.g., skin grafts). Desirably, such therapeutic approaches avoid the non-specific action and unpleasant side effects associated with currently-employed methods. These and other advantages of the present invention, as well as additional inventive features, will be apparent from the accompanying drawing and sequence listing and in the following detailed description.

### DESCRIPTION OF THE DRAWING

FIG. 1 graphically summarizes the application of the inventive method to treat three classes of skin cancers.

### DETAILED DESCRIPTION OF THE INVENTION

The invention provides methods and reagents for epithelial barrier formation and treatment of malignant and benign skin disorders. In some embodiments, the invention employs a Notch agonist, while in other embodiments, the invention employs a Notch antagonist.

A source of a Notch agonist is any exogenous substance that, when provided to the cell or cells in accordance with the inventive method, activates (e.g., agonizes or accentuates the activity of) the Notch signal transduction pathway within the target cell (typically an epithelial cell). Within the context of the present invention, the source of the Notch agonist can activate the Notch pathway either directly or indirectly. The Notch signal transduction pathway is well known in the art and generally involves intracellular activation of a member of the Notch family of signal-transduction receptors. At least four such proteins (Notch1, Notch2, Notch3, and Notch4) are known to exist in mammalian cells, and others also are contemplated by the present invention.

One source of a Notch agonist can be a constitutively active derivative of a Notch protein, either singly or in various combinations of Notch-1, -2,-3,-4, etc. Such protein can be delivered to the target cell intracellularly, such as by transferring an expression cassette encoding the derivative to the target cell, so as to induce the cell to produce the Notch derivative. Such proteins, and nucleic acids, are known in the art (see, e.g., Kopan et al., Development, 120, 2385-96 (1994), Milner et al., Blood, 93, 2431-48 (1996)), and an example of the sequence of one such constitutively active protein is set forth herein as SEQ ID NO:1. Another agonist could be a gamma secretase or a nucleic acid encoding a gamma secretase, the sequences of many which are known in the art.

Another suitable source of a Notch agonist for use in the inventive methods can be a Notch agonist ligand itself (e.g., a substance that is able to activate the Notch signal transduction pathway within cells), such as portions of toporythmic proteins that mediate binding to Notch, and nucleic acids encoding them (which can be administered to express their encoded products in vivo). Many suitable Notch agonist ligands are known in the art, examples of which include Jagged (e.g., Jagged 1 and Jagged 2), Lunatic-Fringe, Manic-Fringe, Radical Fringe, Delta, and Serrate. The sequences of several Notch agonist ligands are set forth herein as SEQ ID NOs:2-8, and others are known in the art (see, e.g., U.S. Pat. Nos. 6,262,025, 6,149,902, 6,004,924, 5,869,282, 5,856,441, 5,849,869, 5,789,195, 5,780,300, 5,750,652, and 5,648,464, each of which is incorporated herein by reference). Where it is desired to apply a Notch agonist ligand, any of these can be selected as desired, as well as active fragments or derivatives of them (e.g., having altered, truncated, or augmented sequences), particularly the DSL domain of Notch ligands. Indeed, a preferred Notch agonist ligand for use in the inventive method is a Jagged 1 derivative, particularly one derived from the Delta/Serrate/LAG-2 domain of hJagged1 (i.e., human Jagged 1), such as is set forth at SEQ ID NO:10.

To facilitate the inventive method, the invention provides a protein having a sequence of amino acids comprising or consisting essentially of one of SEQ ID NOs:9, 10, 11, and 12. Desirably, such protein is a Notch ligand. Of course, any other compound, regardless of its composition, able to activate the Notch pathway upon its exposure to cells expressing a Notch protein can likewise be employed as a Notch agonst within the inventive method. In a preferred embodiment, the agonist is a protein or derivative or fragment thereof comprising a functionally active fragment such as a fragment of a Notch ligand that mediates binding to a Notch protein. In this regard, it is within the skill of the art to generate additional peptide ligands, or non-peptide Notch ligands, for example upon examination of the interaction between Notch ligands (peptides or recombinant proteins) and the soluble Notch ligand binding region.

Where a Notch ligand agonist is provided to the target cell, it can be provided directly (e.g., in a composition comprising the Notch ligand and a physiologically-compatible carrier, such as are described herein). Alternatively, as mentioned, a suitable source of a Notch agonist can act indirectly to activate the Notch pathway within the target cell. For example, agents such as antisense nucleotides targeting endogenous inhibitors or inhibiting pathways that antagonize and or down-regulate the Notch pathway can serve as sources of Notch agonists. In this respect, when such agents are provided to the target epithelial cell, they act to remove endogenous inhibition of Notch signaling within the cell, thus potentiating the Notch pathway. Moreover, as an alternative (or addition) to employing one or more constitutively active form(s) of Notch or Notch agonist ligand(s) directly, a Notch agonist ligand can be provided to the target cell by inducing at least one second cell in the region of the epithelial cell to produce the agonist ligand. For example, the second cell can be induced to produce the Notch agonist ligand by providing at least one chemical agent to the second cell to which it responds by producing the Notch agonist ligand. Alternatively, the second cell can be induced to produce a Notch agonist ligand recombinantly. Thus, an expression cassette encoding at least one Notch agonist ligand can be introduced into the second cell such that it produces the Notch agonist ligand. Inasmuch as many Notch agonist ligands are soluble, where a second cell is induced to provide the Notch agonist ligand, the cell need not be adjacent to the target epithelial cell, although it can be (and typically will be). Indeed, the “second” cell can even be the target epithelial cell, for example where it is desired to auto-activate the Notch pathway within the cell.

In the context of the present invention, a source of a Notch antagonist is an agent that can interfere with the activation of Notch the Notch signal-transduction pathway, for example by blocking or stearically inhibiting the interaction between Notch receptor molecules and their ligands, by scavenging Notch agonists, or by antagonizing the activation of events intracellularly following contact between Notch and a ligand/agonist. Notch antagonists can be proteinaceous or small molecule agents acting extracellularly to modify or bind to Notch or one of its agonists such that activation of the Notch pathway is blocked or attenuated. Other antagonisst can act intracellularly to attenuate production of Notch or one of its agonists (e.g., a nucleic acid antisense to all or a portion of a Notch coding sequence or one of its agonist ligands, or a protein or small molecule interfering with the interaction between Notch and other components of the cellular machinery). Preferred Notch antagonists include gamma secretase inhibitors, many of which are known in the art (see, e.g., Doerfier et al., Proc. Nat. Acad. Sci. (USA), 98(16), 9312-17 (2001); Mizutani et al., Proc. Nat. Acad. Sci. (USA), 98(16), 9026-31 (2001), Evin et al., Biochemistry, 40(28), 8359-68 (2001), Hadland et al., Proc. Nat. Acad. Sci. (USA), 98(13), 7487-91 (2001), Dominguez et al., Amyloid, 8(2), 124-42 (2001), Cutler et al., Prog. Neuropsychopharmacol. Biol. Psychiatry, 25(1), 27-57 (2001), Golde et al. Trends Mol. Med., 7(6), 264-69 (2001); Petit et al., Nat. Cell Biol., 3(5), 507-11 (2001), Moore et al., Ann. N.Y Acad. Sci., 920, 197-205 (2000), Dovey et al., J Neurochem., 76(1), 173-81 (2001), McLendon et al., FASEB J., 14(15), 2383-86 (2000), Shearman Biochemistry, 39(30), 8698-704 (2000), Mumm et al., Mol. Cell, 5(2), 197-206 (2000), Kimberly et al., Nat. Cell Biol., 2(7), 428-34 (2000), Rishton et al., J. Med. Chem., 43(12), 2297-99 (2000), Li et al., Nature, 405(6787), 689-94 (2000), Durkin et al., J. Biol. Chem., 274(29), 20499-504 (1999), Urnoneit et al., Prostaglandins Other Lipid Mediat., 55(5-6), 331-43 (1998), Wolfe et al., J. Med. Chem., 41(1), 6-9 (1998)). Other types of Notch antagonists can be peptide fragments, such as a fragment of Notch able to bind Notch ligands and thus scavenge such ligands or compete with actual Notch receptors for such agonists (e.g., SEQ ID NOs:15-18).

Where a Notch antagonist is provided to the target cell, it can be provided directly (e.g., in a composition comprising the Notch antagonist and a physiologically-compatible carrier, such as are described herein). Alternatively, as mentioned, a suitable source of a Notch antagonist can act indirectly to attenuate the Notch pathway within the target cell. For example, agents such as antisense nucleotides targeting endogenous Notch agonists or potentiating pathways that antagonize and or down-regulate the Notch pathway can serve as sources of Notch antagonists. In this respect, when such agents are provided to the target epithelial cell, they can act to accentuate endogenous inhibition of Notch signaling within the cell, thus antagonizing the Notch pathway. Alternatively, they can act as exogenous inhibitors of the Notch pathway. Moreover, as an alternative (or addition) to supplying one or more notch antagonists directly, a Notch antagonist can be supplied by inducing at least one second cell in the region of the epithelial cell to produce the antagonist, much as discussed above in connection with indirect supply of the agonist.

In accordance with the inventive method, sources of Notch agonists or antagonists are supplied either as small molecule or protein preparations or via gene transfer technology. As mentioned above, protein sequences of many such factors are known, and their genes have been cloned. While any such wild-type proteinaceous factor can be used in the context of the present invention, it can alternatively be or comprise a derivative of a wild-type protein (e.g., an insertion, deletion, or substitution mutant, an active proteolytic cleavage product, etc.). Preferably, any substitution mutation is conservative in that it minimally disrupts the biochemical properties of the agonist or antagonist. Thus, where mutations are introduced to substitute amino acid residues, positively-charged residues (H, K, and R) preferably are substituted with positively-charged residues; negatively-charged residues (D and E) preferably are substituted with negatively-charged residues; neutral polar residues (C, G, N, Q, S, T, and Y) preferably are substituted with neutral polar residues; and neutral non-polar residues (A, F, I, L, M, P, V, and W) preferably are substituted with neutral non-polar residues.

Some protocols of the inventive method call for a given proteinaceous factor (e.g., Notch agonist or antagonist) to be provided directly to the cell as protein. The protein for use in such protocols can be produced by any suitable method. For example, the protein can be synthesized using standard direct peptide synthesizing techniques (Bodanszky, Principles of Peptide Synthesis (Springer-Verlag, Heidelberg: 1984)), such as via solid-phase synthesis (see, e.g., Merrifield, J. Am. Chem. Soc., 85, 2149-54 (1963); Barany et al., Int. J. Peptide Protein Res., 30, 705-739 (1987); and U.S. Pat. No. 5,424,398). Alternatively, a gene encoding the desired protein can be subcloned into an appropriate expression vector using well known molecular genetic techniques. The protein can then be produced by a host cell and isolated therefrom. Any appropriate expression vector (see, e.g., Pouwels et al., Cloning Vectors: A Laboratory Manual (Elsevior, N.Y.: 1985)) and corresponding suitable host cells can be employed for production of agonist or antagonist protein. Expression hosts include, but are not limited to, bacterial species, mammalian or insect host cell systems including baculovirus systems (see, e.g., Luckow et al., Bio/Technology, 6, 47 (1988)), and established cell lines such 293, COS-7, C127, 3T3, CHO, HeLa, BHK, etc. Once isolated, the protein is substantially purified by standard methods and provided to the skin within a suitable composition, as herein described.

In other protocols, the source of a Notch agonist or antagonist is provided to the cell through gene transfer technology. Such protocols employ an expression cassette including the appropriate gene. In addition to the desired coding sequence, the expression cassette also includes a promoter able to drive the expression of the gene within cells associated with target cell. Many viral promoters are appropriate for use in such an expression cassette (e.g., retroviral ITRs, LTRs, immediate early viral promoters (IEp) (such as herpesvirus IEp (e.g., ICP4-IEp and ICPO-IEp) and cytomegalovirus (CMV) IEp), and other viral promoters (e.g., late viral promoters, Rous Sarcoma Virus (RSV) promoters, and Murine Leukemia Virus (MLV) promoters)). Other suitable promoters are eukaryotic promoters, such as enhancers (e.g., the rabbit β-globin regulatory elements), constitutively active promoters (e.g., the β-actin promoter, etc.), signal specific promoters (e.g., inducible and/or repressible promoters, such as a promoter responsive to tetracycline, TNF, RU486, the metallothioneine promoter, etc.), and skin-specific promoters, such as keratin promoters for epidermal expression (Blessing et al., Genes. Devel., 7, 204-15 (1993); Blessing et al., J. Cell. Biol., 135, 227-39 (1993); Byrne et al., Mol., Cell. Biol., 13, 3176-90 (1993)) tie-2 or von Willebrand factor promoters for endothelial expression (Korhonen et al., Blood, 86, 1828-35 (1995); Schlaeger et al., Proc. Nat. Acad. Sci. (USA), 94, 3058-63 (1997)), collagen α1(I) or FSP1 promoters for mesenchymal expression (Okada et al., Am. J. Physiol., 275, F306-14 (1998)), etc.

Within the expression cassette, the desired gene and the promoter are operably linked such that the promoter is able to drive the expression of the gene. As long as this operable linkage is maintained, the expression cassette can include more than one gene (e.g., multiple agonists or antagonists for a potentially synergistic effect). Furthermore, the expression cassette can optionally include other elements, such as polyadenylation sequences, ribosome entry sites, transcriptional regulatory elements (e.g., enhancers, silencers, etc.), or other sequences for enhancing the stability of the vector or transcript, or the translation or processing of the desired transcript within the cells (e.g., secretion signals, leaders, etc.).

For use in the inventive method, the desired expression cassette must be introduced into the cells in a manner suitable for them to express the gene contained therein. Any suitable genetic vector can be employed to introduce the expression cassette into the cells, many of which are known in the art. Examples of such vectors include naked DNA vectors (such as oligonucleotides or plasmids), viral vectors such as adeno-associated viral vectors (Bems et al., Ann. N.Y. Acad. Sci., 772, 95-104 (1995)), adenoviral vectors (Bain et al., Gene Therapy, 1, S68 (1994)), herpesvirus vectors (Fink et al., Ann. Rev. Neurosci., 19, 265-87 (1996)), packaged amplicons (Federoff et al., Proc. Nat. Acad. Sci. (USA), 89, 1636-40 (1992)), papilloma virus vectors, picornavirus vectors, polyoma virus vectors, retroviral vectors, SV40 viral vectors, vaccinia virus vectors, and other vectors. In addition to the expression cassette of interest, the vector also can include other genetic elements, such as, for example, genes encoding a selectable marker (e.g., β-gal or a marker conferring resistance to a toxin), a pharmacologically active protein, a transcription factor, or other biologically active substance.

The vector harboring the expression cassette is introduced into the cells by any method appropriate for the vector employed. Many such methods are well-known in the art. Thus, plasmids or oligonucleotide vectors are transferred by methods such as calcium phosphate precipitation, electroporation, liposome-mediated transfection, gene gun, microinjection, viral capsid-mediated transfer, polybrene-mediated transfer, protoplast fusion, etc. Viral vectors are best transferred into the cells by infecting them; however, the mode of infection can vary depending on the virus.

Cells into which the desired gene or genes have been transferred can be used in the inventive method as transient transductants. Alternatively, where the cells are cells in vitro, they can be subjected to several rounds of clonal selection (if the vector also contains a gene encoding a selectable marker, such as a gene conferring resistance to a toxin) to select for stable transformants. Within the cells, the gene is expressed such that the cells produce the desired product (e.g., protein or, in some cases, non-translated RNA). Successful expression of the gene can be assessed via standard molecular biological techniques (e.g., Northern or Western blotting, immunoprecipitation, enzyme immunoassay, etc.).

However it is supplied, by providing the source of the Notch agonist to an epithelial cell, the Notch Pathway is activated within it to induce differentiation of the epithelial cell. Accordingly, in one embodiment, the invention provides a method of inducing differentiation of an (i.e., at least one) epithelial cell. In accordance with this aspect of the invention, an exogenous source of a Notch agonist is supplied to the epithelial cell. The epithelial cell can be any cell found within any type of epithelial tissue or in association with surrounding cell types (e.g., dermal cells, subdermal cells, melanocytes, glandular cells, cells of polarized structures (e.g., hair, feathers, scales, etc.). The cells also can be can be employed to differentiate some skin-associated non-epithelial cells, such as immunocytes and monocyte-derived dendritic cells.

Terminal differentiation of epithelial cells can be assessed by any appropriate method, many of which are known in the art. Thus, for example, where the epithelial cell is a keratinocyte, its differentiation can be monitored by assaying for the production of certain markers, such as keratin 1, involucrin, loricrin, appearance of keratohyalin granules, activation of IKKα/NF-κB, etc. (see, e.g., Yuspa et al., J. Cell. Biol., 109, 1207-17 (1989); Green, Cell, 11, 405-16 (1977)). Another mark of terminal differentiation, particularly where more than one cell is affected and it is within an epithelial structure (e.g., an intact or partial epithelial tissue) is the formation of gross structures associated with differentiated epithelial cells, particularly barrier structures.

In many applications in which the method is applied to cutaneous tissue, the targeted epithelial cell typically is a keratinocyte or some other cell within cutaneous epithelial tissue (or epidermal equivalent). However, in other embodiments, the cell can be within extracutaneous epithelium (e.g., oral mucosal, cornea, gastrointestinal epithelia, urogenital epithelia, respiratory epithelia, etc.), and in other applications, the cell can be a malignant or pre-malignant cell, as is described herein.

The inventive method can be employed in various contexts depending on its desired end use. Thus, the inventive method can be employed to promote formation of a barrier within epithelium (i.e., epithelial tissue). In accordance with this aspect of the invention, an exogenous source of a Notch agonist is delivered to epithelial cells within the epithelial tissue such that they are induced to form a barrier within the epithelium. Formation of such a barrier can be assessed by histological examination of the tissue. For example, where the barrier is stratum corneum, its appearance in cutaneous epithelium will be readily apparent to one of skill in the art.

Regardless of the desired application (e.g., clinical or research) of the inventive method, in one mode of application, it can be applied to an epithelial cell ex vivo. In one such ex vivo application, the invention provides a method for producing differentiated epidermis. In accordance with this aspect of the invention, undifferentiated (or partially differentiated) epidermal tissue is cultured in the presence of a source of a Notch agonist such that the Notch Pathway is activated within at least one cell of the cultured epidermal tissue so as to induce differentiation of the epidermis. The tissue is cultured for a time sufficient to result in formation of mature epidermal structure, such as the presence of a barrier and substratum. Desirably, the tissue is cultured by being submerged in a solution (preferably a conventional defined medium) containing the source of a Notch agonist. This application of the method can be used alone or adjunctively to culture epidermal tissues for subsequent implantation into patients (e.g., skin or other epithelial grafts).

In other applications, the method can be employed on an epithelial cell in vivo. In such applications, the method can be used alone or adjunctively as part of a treatment for any of a number of epithelial disorders, such as those set forth above. For use in vivo, a protein source of a Notch agonist, or a gene transfer vector encoding it, is incorporated into a suitable solution including a carrier and, as appropriate, a suitable mode of delivery for in vivo application. In certain embodiments of the present invention compositions comprising compounds capable of activating the Notch pathway are administered to a patient. The composition can be dissolved or dispersed in a pharmaceutically acceptable carrier or aqueous medium. Aqueous compositions of gene therapy vectors expressing Notch receptor/ligands, mutants thereof, or antisense nucleic acids are also contemplated. The phrases “pharmaceutically or pharmacologically acceptable” refer to the molecular entities and compositions that do not produce an adverse, allergic or other untoward reaction when administered to an animal, or a human, as appropriate.

For clinical use, the method can be employed to treat patients suffering from a variety of disorders associated with barrier formation, e.g., bun victims, plastic surgery patients, or those suffering from keratosis, aged skin, alopecia (e.g., androgenic alopecia, alopecia areata, etc.), asteototic skin (dry skin, winter itch), Bowen's disease, cancers (e.g., keratocanthoma, squamous cell carcinoma, basal cell carcinoma, etc.), dermatitis (e.g., atopic dermatitis, allergic/irritant contact dermatitis, etc.), drug reactions, ichthyotic skin, photodamaged skin, psoriasis, sunburn, incontinentia pigmenti, basal and squamous cell carcinoma, and the like. The method can also be used to induce the differentiation of mucosal epithelium in vivo (e.g., pulmonary epithelium, gastrointestinal (GI) epithelium, etc.), and can be used to treat patients suffering from malformation of such barriers, such as smokers, smoke inhalation victims, and those suffering from acute respiratory distress syndrome (ARDS), respiratory distress syndrome (RDS), cystic fibrosis, and others.

Aside from being applicable to a variety of epithelial cell types, the method can be employed to treat the cells in a variety of conditions. Thus, for example, the invention provides a method of retarding the progression of a pre-malignant condition towards epithelial malignancy. In such method, a source of a Notch agonist is provided to at least one pre-malignant epithelial cell. As such, the Notch pathway is activated within the cell so as to retard progression of the cell towards malignancy. In this context, progression towards malignancy is retarded if the cell's progression towards malignant phenotype is slowed in any respect (e.g., cessation or slowing of cell cycling). Desirably, however, activating the Notch pathway can prevent a pre-malignant epithelial cell from undergoing malignant transformation. Of course, as discussed above, the method also can induce the pre-malignant epithelial cell to terminally differentiate, which is inconsistent with malignant progression. Where a pre-malignant condition involves more than one pre-malignant epithelial cell, the method can prove effective even if a subset of the pre-malignant cells is retarded towards malignancy or prevented from undergoing malignant transformation. However, the method desirably acts on all treated pre-malignant cells.

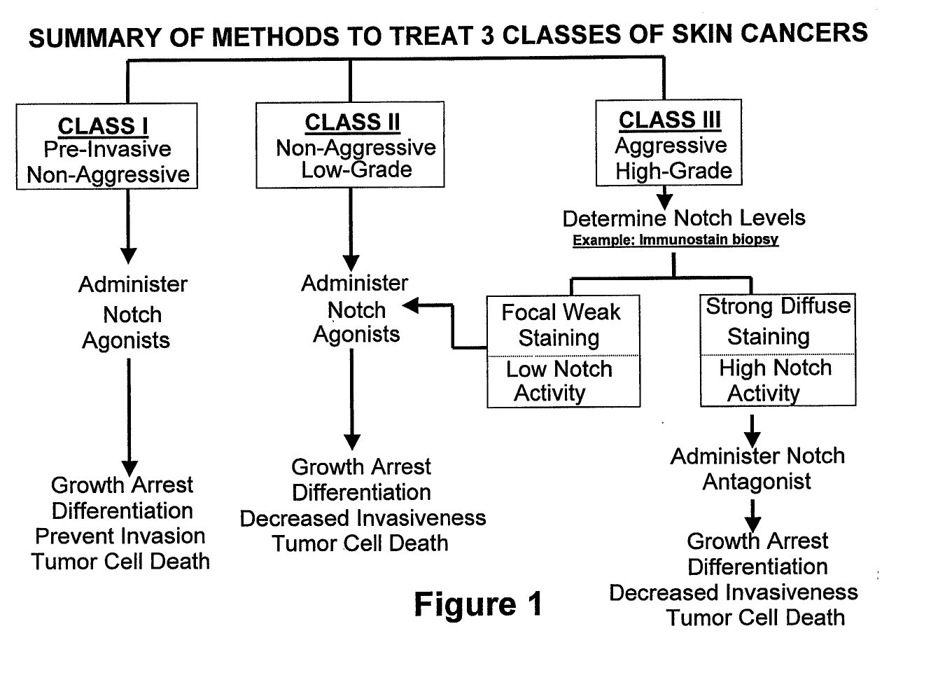

In another embodiment, the invention provides a method of treating a cutaneous malignancy (skin cancer) that has already developed by retarding its progression. In accordance with this embodiment, an agonist or antagonist of the Notch pathway is administered to the skin cancer, whereby upon contact with the agonist or antagonist, the progression of the skin cancer is retarded. The cancer can be of any type (e.g., CTCL, melanoma, or squamous or basal cell carcinoma) or grade (non-invasive, low-grade, or aggressive); however, the grade of cancer determines whether the appropriate treatment employs a Notch agonist or an antagonist. In this regard, a careful diagnosis of the grade of skin cancer should be undertaken prior to or in conjunction with the application of the inventive method to treat skin cancer. In this regard, where the cancer is non-aggressive (e.g., pre-invasive or low-grade carcinoma or melanoma, or non-aggressive or non-leukemic CTCL (indicated generally as Class I and Class II in FIG. 1), or, when biopsied, does not display strong overexpression (e.g., exhibits weak or focal staining) of Notch proteins or their ligands, it is appropriate to employ a Notch agonist in the treatment of such cancers (see FIG. 1). Without being bound by any particular theory, it is suggested that the cells of such non-aggressive or pre-invasive stage skin cancers respond to Notch signals in a similar manner as non-cancerous epithelial cells such that Notch activation potentiates the cells towards a differentiated phenotype. Thus, Notch activation of the cells of such cancers can induce differentiation of the cancerous cells, which is inconsistent with a malignant phenotype, or can at least attenuate the de-differentiation associated with malignant proliferation. Conversely, where the cancer is diagnosed as aggressive or when biopsied, displays general overexpression of Notch proteins or their ligands (e.g., strong or diffuse staining), it is more appropriate to employ a Notch antagonist in the treatment of such cancers (see FIG. 1, Class III). Again without being bound by any particular theory, it is believed that Notch overexpression contributes to the aggressive behavior of such cancers, which have progressed beyond the point at which they can respond to Notch activation by differentiating as other epithelial cells.

However treated, cells of the cancer can be in any location in vivo (e.g., within the skin of a patient (e.g., within a lesion or tumor) or metastatic; however, the non-invasive cancers typically are within the skin and not metastatic. Moreover, the cancerous cells can be in vitro, such as a cell line derived from skin cancer cells. Where the cells (i.e., the skin cancer) are in vivo, the agonist or antagonist can be administered to the patient having the cancer in any manner appropriate to deliver the antagonist to the cells of the cancer.

Successful application of the method retards the progression of the skin cancer, which can be assessed by several methods. For example, where the cancer is aggressive CTCL, melanoma, or carcinoma, successful application of the method can result in the reversion of cells within the cancer to a non-aggressive phenotype. In this regard, a non-aggressive phenotype can be assessed in vitro, for example by assaying the capacity of the cells to penetrate real or artificial tissue matrices. The cells also can be assayed for any other standard phenotypic hallmark of aggressive or non-aggressive phenotype, or for overexpression of Notch receptors or ligands, as discussed herein. In another example, where the cancer is aggressive CTCL, the method results in the cessation of the progression of cells within the CTCL towards a leukemic phenotype, which can be correlated with the degree to which the cancer exhibits epidermotropism. The method can result in retarded cancer progression to any measurable extent; it is not necessary for all of the cancer progression to be retarded. However, in some patients, the method can effect a nearly complete halt in further progression of the skin cancer, and possibly even a regression of the cancer to a less aggressive, or even non-malignant state. It is also possible that the method can result in growth arrest, differentiation, decreased or blocked invasion (e.g., of basal membrane), and even death of the cancerous cells. It is to be understood, however, that any degree to which the inventive method results in retarded cancer development is of great potential benefit to the patient, as it can afford the patient a longer time to combat the cancer by other methods, thus increasing the likelihood that the cancer can be cured or excised completely.

In conjunction with the aspects of the invention discussed above, a source of Notch agonist or antagonist is delivered to cells within or near an epithelium or epidermis. Where the cells are in vivo, the source of such an agent can be formulated into a pharmaceutical composition (or “medicament”) comprising the source of such agent (or, in some applications, the agent (i.e., Notch agonist or antagonist)) and a pharmaceutically or pharmacologically-acceptable carrier. Such carriers include any and all solvents, dispersion media, coatings, antibacterial and antifungal agents isotonic, and absorption delaying agents, and the like. The use of such media and agents for pharmaceutical active substances is well known in the art. Except insofar as any conventional media or agents are incompatible with the active ingredient, its use in the therapeutic compositions is contemplated. Supplementary active ingredients can also be incorporated into the compositions. Thus, one aspect of the invention provides the use of a Notch agonist or antagonist to prepare a medicament for differentiating an epithelial cell, for inducing epithelial barrier formation, for treating disorders associated with disrupted epithelial barriers, or for treating malignancies as herein described. The formulation of such medicament, of course, depends on the desired mode of treatment, including the location of the tissue or cells, and the condition of the tissue.

For human administration, preparations should meet purity, potency, sterility, pyrogenicity, moisture content, and general safety standards as required by applicable regulations (e.g., the U.S. FDA Center for Drug Evaluation and Research Center for Biologics Evaluation and Research or similar agencies in other jurisdictions). The biological material should be extensively dialyzed to removed undesired small molecular weight molecules and/or lyophilized for more ready formulation into a desired vehicle, where appropriate. The active compounds then will generally be formulated for parenteral administration, e.g., formulated for injection via the intravenous, intramuscular, subcutaneous, intralesional, or even intraperitoneal routes.

The preparation of an aqueous composition that contains a compound capable of altering the activation of the Notch pathway (e.g., a Notch agonist or antagonist) as an active component or ingredient will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified. The pharmaceutical forms suitable for injectable use include sterile aqueous solutions or dispersions; formulations including sesame oil, peanut oil or aqueous propylene glycol; and sterile powders for the extempraneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

A protein, polypeptide, antibody, agonist or antagonist for use in the methods of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be solvent or dispersion medium containing, for example, water, ethanol, polyol (for example, glycerol, propylene glycol, a liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredients plus any additional desired ingredient from a previously sterile-filtered solution thereof.

In terms of using polypeptide therapeutics as active ingredients, the technology of U.S. Pat. Nos. 4,608,251; 4,601,903; 4,559,231; 4,559,230; 4,596,792; and 4,578,770 each incorporated herein by reference, can be used.

The preparation of more, or highly, concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitable buffered if necessary and the liquid diluent first rendered isotonic with sufficient saline or glucose. These particular aqueous solutions are especially suitable for intravenous, intramuscular, subcutaneous and intraperitoneal administrations. In this connection, sterile aqueous media which can be employed will be known to those of skill in the art of light of the present disclosure. For example, on dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The active polypeptides or agents can be formulated for parenteral administration, such as intravenous or intramuscular injection, other pharmaceutically acceptable forms include, e.g., tablets or other solids for oral administration; liposomal formulations; time release capsules; and any other form currently used, including cremes.

One can also use nasal solutions or sprays, aerosols or inhalants in the present invention. Nasal solutions are usually aqueous solution designed to be administrated to the nasal passages in drops or sprays. Nasal solutions are prepared so that they are similar in many respects to nasal secretions, so that normal ciliary action is maintained. Thus, the aqueous solution usually are isotonic and slightly buffered to maintain a pH of 5.5 to 6.5.

In addition, antimicrobial preservatives, similar to those used in ophthalmic preparations, and appropriate drug stabilizers, if required, can be included in the formulation. Various commercial nasal preparations are known and include, for example, antibiotics and antihistamines and are used for asthma prophylaxis.

Additional formulations which are suitable for other modes of administration include vaginal suppositories and pessaries. A rectal pessary or suppository can also be used.

Suppositories are solid dosage forms of various weights and shapes, usually medicated, for insertion into the rectum, vagina or the urethra. After insertion, suppositories soften, melt or dissolve into the cavity fluids.

In general, for suppositories, traditional binders and carriers can include, for example, polyalkylene glycols or triglycerides; such suppositories can be formed from mixtures containing the active ingredient in the range of 0.5% to 10%, preferable 1%-2%.

Vaginal suppositories or pessaries are usually globular or oviform and weighing about 5 g each. Vaginal medications are available in a variety of physical forms, e.g., creams, gels or liquids, which depart from the classical concept of suppositories. Vaginal tablets, however, do meet the definition, and represent convenience both of administration and manufacture.

In certain defined embodiments, oral pharmaceutical compositions will comprise an inert diluent or assimible edible carrier, or they can be enclosed in hard or soft shell gelatin capsule, or they can be compressed into tablets, or they can be incorporated directly with the food of the diet. For oral therapeutic administration, the active compounds can be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations can, of course, be varied and can conveniently be between about 2 to about 75% of the weight of the unit, or preferably between 25-60%. The amount of active compounds in such therapeutically useful composition is such that a suitable dosage will be obtained.

Tablets, troches, pills, capsules and the like can also contain the following: a binder, as gum tragacanth, acacia, cornstarch, or gelatin, excipients, such as dicalcium phosphate, a disintegrating agent, such as corn starch, potato starch, alginic acid and the like; a lubricant, such as magnesium stearate; and a sweetening agent, such as peppermint, oil of wintergreen, or cheery flavoring. When the dosage unit is a capsule, it can contain, in addition to material of the above type, a liquid carrier. Various other materials can be present as coatings or to otherwise modify the physical form of the dosage unit. For instance, tablets, pills, or capsules can be coated with shellac, sugar, or both. A syrup or elixir can contain the active compounds sucrose as a sweetening agent and parabens as preservatives, a dye and flavoring, such as cherry flavor.

Although essentially any method of delivery of a compound that activated the Notch Pathway can be used, in embodiments of treating disorders of the skin it is preferred that the compound is delivered transdermally. Thus, the invention provides a transdermal patch comprising a reservoir containing a compound that activates the Notch receptor and its respective ligand in an non-aqueous matrix. The patch can be placed directly to the area of epithelium to which it is desired to employ the inventive method.

A prerequisite for the development of transdermal delivery system of a compound that activates the Notch Pathway is, however, that the compound that activates the Notch receptor or its respective ligand is capable of penetrating the skin at a sufficiently high rate and is not metabolized during the precutaneous absorption. Methods and compositions for producing medicaments for transdermal delivery are well known to those of skill in the art. The patents listed above have been incorporated herein by reference to provide guidance for making such transdermal medicaments.

The actual administration or use of the transdermal compositions according to the present invention can be in any conventional form and can follow any of the methods generally known in the art. For instance, a compound that activated the Notch receptor and its respective ligand can be used in association with any pharmaceutical dosage form such as, for example, by not limited thereto, and solution, ointment, lotion, paste, jelly, gel, cream, spray or aerosol generally known in the art. As such, a compound that activates the Notch receptor and its respective ligand in association with the pharmaceutical dosage form can be used directly as a topical composition or used in combination with an additional drug delivery service, for example, but not limited thereto, patches, gauze, compresses, or the like, again, as generally known in the art. The dosage forms can contain any type of absorption or permeation enhancers such as fatty acids, fatty acid esters and fatty alcohols or any other non-toxic compounds which are known to increase skin permeability. In particular, the transdermal compositions, can be administered in the form of a patch wherein, a compound that activates the Notch receptor and its respective ligand is present in a polymeric matrix or in a reservoir system combined with a polymeric rate controlling membrane.

“Permeation enhancer” or “absorption enhancer” as used herein relates to any agent that increases the permeability of skin to a compound that activates the Notch receptor and its respective ligand, i.e., so as to increase the rate at which a compound that activates the Notch receptor and its respective ligand penetrates through the skin and enters the bloodstream. The enhanced permeation effected through the use of such enhancers can be observed by measuring the rate of diffusion of drug through animal or human skin using a diffusion cell apparatus. Such devices are well known to those of skill in the art (see for example U.S. Pat. No. 5,906,830).

U.S. Pat. No. 5,906,830 (incorporated herein by reference) describes methods for manufacturing transdermal drug delivery systems containing supersaturated drug reservoirs, such that higher drug fluxes are obtained. These methods involve heating the drug reservoir components to a predetermined temperature. Generally, this temperature is higher than the depressed melting temperature of the polymer-drug admixture which will serve as the drug reservoir. It is contemplated that the methods described therein will be useful for producing the transdermal delivery medicaments of the present invention.

U.S. Pat. No. 4,409,206 relates to a method for preparing transdermal drug delivery systems containing the active substance in an amorphous form. Initially, a polyarylate film is prepared by solvent casting. A drug solution or suspension is then applied to the film and the solvent is removed by evaporation. U.S. Pat. No. 4,746,509 describes transdermal medicaments with the active ingredient dispersed in a drug reservoir in crystalline and/or solubilized form. Another method that can be useful in the present invention is described in U.S. Pat. No. 4,832,953 in which the drug delivery systems containing liquid drugs capable of forming crystalline hydrates are formed. U.S. Pat. No. 4,883,669 describes a transdermal drug delivery system in which the drug is microdispersed in a polymeric matrix disc later which serves as the drug reservoir. U.S. Pat. No. 5,332,576 describes preparation of compositions for topical application, in which the drug is added to certain components, not including the bioadhesive carrier, and then heated at a temperature in the range of about 70° C. to 90° C. until all of the drug is dissolved. After the solution is cooled, the bioadhesive is added and the composition is applied to a backing material. It is contemplated that such methods of producing a transdermal delivery system can be used herein.

In addition, transdermal delivery forms can further include diffusional systems and dissolution systems. In diffusional systems, the release rate of drugs is further effected by its rate of diffusion through a water-insoluble polymer. There are generally two types of diffusional devices, reservoir devices in which a core of drug is surrounded by polymeric membrane; and matrix devices in which dissolved or dispersed drug is distributed substantially uniformly and throughout an inert polymeric matrix. In actual practice, many systems that utilize diffusion can also rely to some extent on dissolution to determine the release rate.

Common practices utilized in developing transdermal delivery devices with reservoir systems include microencapsulation of drug particles (e.g., see U.S. Pat. No. 5,814,599). Frequently, particles coated by microencapsulations form a system where the drug is contained in the coating film as well as in the core of the microcapsule. As such, in transdermal delivery, such a microcapsule can be applied as a microemulsion or be coated onto a patch type delivery system. Drug release typically includes a combination of dissolution and diffusion with dissolution being the process the controls the release rate. Common material used as the membrane barrier coat, alone or in combination, include but are not limited to, hardened gelatin, methyl and ethyl-cellulose, polyhydroxymethacrylare, polyvinylacetate, and various waxes.

The most common method of microencapsulation is coacervation, which involves addition of a hydrophilic substance to a colloidal dispersion. The hydrophillic substance, which operates as the coating material, is selected from a wide variety of natural and synthetic polymers including shellacs, waxes, starches, cellulose acetates, phthalate or butyrate, polyvinyl-pyrrolidone, and polyvinyl chloride. After the coating material dissolves, the drug inside the microencapsule is immediately available for dissolution and absorption. Drug release, therefore, can be controlled by adjusting the thickness and dissolution rate of the coat. For example, the thickness can be varied from elss than one μm to 200 μm by changing the amount of coating material from about 3 to 30 percent by weight of the total weight. By employing different thicknesses, typically three of four, the active agent will be released at different, predetermined times to afford a delayed release affect.

In matrix systems, three major types of material are frequently used in the preparation of the matrix systems which include insoluble plastics, hydrophilic polymers, and fatty compounds. Plastic matrices which have been employed include methyl acrylate-methyl methacrylate, polyvinyl chloride and polyethylene. Hydrophilic polymers include methyl cellulose, hydroxypropylcellulose, hydroxpropyl-ethylcellulose, and its derivatives and sodium carboxy-methycellulose. Fatty compounds include various waxes such as carnauba wax, and glyceryl tristearate. Preparation of these matrix systems are by methods well known to those skilled in the art. These methods of preparation generally comprise mixing the drug with the matrix material and compressing the mixture into a suitable pharmaceutical layer. With wax matrixes, the drug is generally dispersed in molten wax, which is then congealed, granulated and compressed into cores.

Approaches to further reducing the dissolution rate include, for example, coating the drug with a slowly dissolving material, or incorporating the drug into a formulation with a slowly dissolving carrier. Encapsulated dissolution systems are prepared either by coating particles or granules of drug with varying thickness or slowly soluble polymers or by microencapsulation.

Matrix dissolution systems are prepared by compressing the drug with a slowly dissolving polymer carrier into formulation suitable for transdermal delivery such as gel matrix, a cream, a colloid, an ointment, a lotion or any other suitable form of topical transdermal delivery. Generally there are two methods for preparing the drug-polymer particles, congealing and aqueous dispersion methods. In the congealing method, the drug is mixed with a polymer or wax material and either cooled or cooled and screened or spray-congealed. In the aqueous dispersion method, the drug-polymer mixture is simply sprayed or placed in water and the resulting particles are collected.

Osmotic systems are also available where osmotic pressure is employed as the driving force to afford release of a drug. Such systems generally consist of a core of drug surrounded by a semi-permeable membrane containing one or more orifices. The membrane allows diffusion of water into the core, but does not allow release of the drug except through the orifices. Examples of materials used as the semi-permeable membrane include polyvinyl alcohol, polyurethane, cellulose acetate, ethylcellulose, and polyvinyl chloride.

In a particularly preferred embodiment, it is contemplated that topical dosage forms are in the form of a compound that activates the Notch receptor and its respective ligand-containing patch. It is contemplated that such a patch comprises a matrix type or reservoir type patch system containing a compound that activates the Notch receptor and its respective ligand in combination with a penetration enhancing delivery device/process such as iontophoresis, electroporation or ultrasound. Reservoir type patch systems and iontophoresis are both well known for transdermal delivery. Accordingly, it is contemplated that the use of these transdermal systems afford the appropriate permability coefficients and fluxes of a compound that activates the Notch receptor and its respective ligand through a predetermined area of mammalian skin tissue. These coefficients and fluxes are preferably established as being sufficient in magnitude to be practical for producing time-sustained dosage rates consistent for the therapeutic effects of a compound that activates the Notch receptor and its respective ligand over prolonged periods of time.

By “predetermined area of mammalian skin tissue” is intended to refer to a defined area of intact unbroken living skin or mucosal tissue. That area will usually be in the range of about 5 cm² to about 100 cm², more usually in the range of about 20 cm² to about 60 cm². However, it will be appreciated by those skilled in the art of transdermal drug delivery that the area of skin or mucosal tissue through which drug is administered can vary significantly, depending on patch configuration, dose and the like.

A patch device generally comprises a laminated composite of a drug reservoir, a backing layer which serves as the upper surface of the device during use and is substantially impermeable to the drug, and a release liner to protect the basal surface of the device prior to use. Optionally, a contact adhesive layer or a peripheral ring of contact adhesive can be provided on the basal surface of the device to enable adhesion of the device to the skin during drug delivery.

The backing layer functions as the primary structural element of the device and provides the device with much of its flexibility, drape and, preferably, occlusivity. The material used for the backing layer should be inert and incapable of absorbing drug, enhancer or other components of a composition comprising a compound that activates the Notch receptor and its respective ligand contained within the device. The backing is preferably made of one or more sheets or films of a flexible elastomeric material that serves as a protective covering to prevent loss of drug and/or vehicle via transmission through the upper surface of the device, and will preferably impart a degree of occlusivity to the device, such that the area of the skin covered on application becomes hydrated. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. Examples of materials useful for the backing layer are polyesters, polyethylene, polypropylene, polyurethanes and polyether amides. The layer is preferably in the range of about 15 μm to about 250 μm in thickness, and can, if desired, be pigmented, metallized, or provided with a matte finish suitable for writing.

The reservoir layer component of the transdermal patch can double as the means for containing a compound that activates the Notch receptor and its respective ligand-containing formulation and as an adhesive for securing the device to the skin during use. That is, as the release liner is removed prior to application of the device to the skin, the reservoir layer serves as the basal surface of the device which adheres to the skin.

Suitable polymeric materials for the drug reservoir include pressure-sensitive adhesives which are physically and chemically compatible with the drug to be administered, and the carriers and vehicles employed. Such adhesives include, for example, polysiloxanes, polyisobutylenes, polyacrylates, polyurethanes, plasticized ethylene-vinyl acetate copolymers, low molecular weight polyether amide block polymers (e.g., PEBAX), tacky rubbers such as polyisobutene, polystyrene-isoprene copolymers, polystyrene-butadiene copolymers, and mixtures thereof. In addition, other adhesive materials for use as reservoir layer include acrylates, silicones and polyisobutylenes, combinations of acetate-acrylate copolymers (such as can be obtained under the trademarks GELVA® 737 and GELVA® 788 form Monsanto Chemical Co.) with a water soluble, water-absorptive polymer such as polyvinyl alcohol, gelatin, polyacrylic acid, sodium polyacrylate, methylcellulose, carboxymethylcellulose, polyvinylpyrrolidone, gum acacia, gum tragacanth, carrageenan and guar gum, particularly polyvinylpyrrolidone.

The reservoir layer is comprised of adhesive material as described above, and will generally range in thickness from about 10 to about 300 μM, preferably approximating 75 μM. Alternatively, the adhesive layer can form a separate and distinct portion of the device, for example, the adhesive portion of the device can comprise an annulus around the reservoir layer, this annulus serving to ensure that the patch device adheres to the skin. Similarly, the reservoir area and the adhesive area can be sandwiched between each other as strips or annular portions, for example a patch is contemplated in which there are stripes of drug reservoir separated by stripes of adhesive.

The release liner is a disposable element which serves only to protect the device prior to application. Typically, the release liner is formed from a material impermeable to the drug, vehicle and adhesive, and which is easily stripped from the contact adhesive. Release liners are typically treated with silicone or fluorocarbons.

Any of these transdermal drug delivery devices can also be provided with a release rate controlling membrane to assist in controlling the flux of drug and/or vehicle from the device. Such a membrane will be present in a drug delivery device beneath and typically immediately adjacent to the drug reservoir, and generally between the drug reservoir itself and the adhesive layer which affixes the device to the skin. Representative materials useful for forming rate-controlling membranes include polyolefins such as polyethlene and polypropylene, polamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

In certain embodiments, the use of liposome and/or nanoparticles is contemplated for the introduction of antibodies, polypeptides or agents, or gene therapy vectors, including both wild-type and antisense vectors, into host cells. The formation and use of liposomes is generally known to those of skill in the art, and is also described below.

Nanocapsules can generally entrap compounds in a stable and reproducible way. To avoid side effects due to intracellular polymeric overloading, such ultrafine particles sized around 0.1 μm) should be designed using polymers able to be degraded in vivo. Biodegradable polyalkyl-cyanoacrylate nanoparticles that meet these requirements are contemplated for use in the present invention, and such particles can be are easily made.

Liposomes are formed from phospholipids that are dispersed in an aqueous medium and spontaneously from multilamellar concentric bilayer vesicles (also termed multilamellar vesicles (MLVs). MLVs generally have diameters from 25 nm to 4 μm. Sonication of MLVs results in the formation of small unilamellar vesicles (SUVs) with diameters in the range of 200 to 500 A, containing an aqueous solution in the core.

The following information can also be utilized in generating liposomal formulations. Phospolipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperature undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars, and drugs.

Liposomes interact with cells via four different mechanisms: Endocyotosis by phagocytic cells of the reticuloendothelial system such as macrophages and neutrophils; absorption to the cell surface, either by nonspecific weak hydrophobic or electrostatic forces, or by specific interactions with cell-surface components; fusion with the plasma cell membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time.

In another embodiment, the invention provides a method of assaying for genetic propensity of a patient to develop a disorder associated with epithelial barrier formation. In accordance with this method, DNA or RNA is obtained from the patient, and a characteristic of the DNA or RNA from the patient encoding a Notch protein or a Notch ligand is deduced. The characteristic can be, for example, a sequence, a restriction pattern, protein binding patterns (e.g., gel-hift assays, RNAse or DNAse protection assays, etc.). Sequencing polynucleic acids is routine in the art. A description of methods of detecting somatic mutations in genes associated with disease is provided by U.S. Pat. No. 6,624,819 (incorporated herein by reference). Such methods include, but are not limited to, PCR-based methods, DNA sequencing, SCCP methods, RNase protection based methods. Methods to determine the expression levels of mRNA or protein are well known to those skilled in the art. These include, but are not limited to, RT-PCR-based methods, within or without real time PCR, ribonuclease protection assays, Northern blotting, insertion hybridization, DNA array hybridization, immunohistochemistry, and immunoasays such as ELISA and Western blotting. Finally, the patient's sequence is compared to the wild-type sequence to determine whether the patient's sequence encodes a protein differing in amino acid sequence from a corresponding wild-type protein. Difference from wild-type is indicative, but does not necessarily prove, a predisposition to contract a disease or disorder of epithelial tissue, such as those set forth herein.

In anther embodiment, the invention provides a diagnostic test to determine the expression levels of Notch ligands, receptors, or other Notch signaling compounds in cells. Typically, such cells are epithelial cells, but they also can be cells of the immune system (including but not limited to malignant lymphocytes) that are present within dermis or submucosa. The expression can be ascertained using standard methods at the level of protein production or mRNA transcripts to determine the level of activity. Deviation from a wild-type expression level (either accentuation or attenuation) can be used to diagnose epithelial disorders and/or to assess their stage and prognosis.

In another embodiment, the invention provides a method of diagnosing aggressive melanoma. In accordance with this method, a tissue biopsy obtained from a patient is assayed for the overexpression of a protein associated with Notch activation. Preferably, the method involves assaying for the overexpression of one or more Notch ligand, Notch receptor, and optionally one or more endothelial cell markers/adhesion molecules. For example, the method can involve the assay of the tissue sample for overexpression of one or more proteins such as JAG-1, JAG-2, and Delta, Notch-1, Notch-2, Notch-3, Notch-4, CD31, CD34, or CD54. Of course, the method can be practiced by assaying for the expression of multiple such factors (i.e., two or more, three or more, four or more, or even all of them), such as within the same tissue sample or even in the same cells of the tissue sample). Overexpression of one or more of the proteins leads to the positive diagnosis of aggressive melanoma within the patient, as opposed to a non-aggressive presentation of melanoma or benign tissue.

In another embodiment, the invention provides a method of diagnosing aggressive CTCL. The method involves obtaining a tissue biopsy from a patient, and assaying the tissue specimen for the overexpression of a T-cell-specific marker (e.g., CD3, CD4, CD8, etc.), a Notch receptor, and a Notch ligand. A staining pattern in which the T-cell-specific marker and Notch receptor are expressed in a first cell, and a Notch ligand is expressed in a second cell distinct from the first is associated with a positive diagnosis of aggressive CTCL, as is strong or diffuse Notch signaling in the biopsy. Desirably, the method is employed with other diagnostic methods, so as to increase accuracy of the diagnosis.

Overexpression of the desired factor(s) can be assessed by any desired method. For example, the level of expression of the desired factor(s) (e.g., assessed though immunostaining) within all or a portion of the tissue sample can be compared to a control tissue, which can be phenotypically normal tissue in the same tissue sample, or can be another tissue sample. A noticeably higher level of expression relative to the control is taken as being overexpression. In this regard, strong, diffuse staining can be interpreted as overexpression (and aggressive carcinoma, melanoma or CTCL), whereas focal or weak staining is not indicative of overexpression (and thus indicative of non-aggressive carcinoma, melanoma or CTCL).

### EXAMPLES

While one of skill in the art is fully able to practice the instant invention upon reading the foregoing detailed description, in conjunction with the drawings and the sequence listing, the following examples will help elucidate some of its features. In particular, they demonstrate the differential expression of Notch receptors and ligands in normal skin, and the inducement of differentiation and barrier formation upon exposure or epithelial structures to sources of Notch agonsts. Of course, as these examples are presented for purely illustrative purposes, they should not be used to construe the scope of the invention in a limited manner, but rather should be seen as expanding upon the foregoing description of the invention as a whole. The methods employed in these examples are as follows:

Tissue Samples

Portions of 3 mm punch biopsies of normal human adult skin skin biopsies were fixed in formalin and paraffin embedded, or were cryopreserved by mounting on gum tragacanth and snap-frozen in liquid nitrogen chilled isopentane, and stored at −80° C.

Cell Culture and Treatments

Keratinocyte cultures were initiated from discarded neonatal foreskins, in which epidermis was separated from dermis using dispase (Mukherjee et al, Chemical and Structural Approaches to Rational Drug Design (Weiner et al., eds.) CRC Press, pp. 237-61 (Boca Raton, 1994). Keratinocytes were induced to proliferating using a low calcium (0.07 mM), serum-free medium (KGM, Clonetics Corp., San Diego, Calif.). Keratinocytes were grown in 10 cm plastic petri dishes, and passaged at 60-70% confluency, and maintained in a humidified incubator at 37° C. with 5% CO₂. Keratinocytes were also grown in 8-well Lab Tek Chambers as previously described (Mukherjee et al., supra). Keratinocytes were used between 2 and 4 passages. In some experiments, keratinocytes were pre-treated with KGM containing an elevated calcium ion concentration (i.e. 2 mM) for 24 hrs prior to addition of JAG-1 peptide, or other stimuli as indicated.

To perform protein analysis on keratinocytes from different layers of the skin, thin keratome samples (1 mm thick) of normal human skin were treated with trypsin (0.05%, 1 hr at 37° C.) and the single cell suspension transferred to a discontinuous Percoll gradient (see, e.g., Qin et al., J. Biol. Chem., 274, 37957-64 (1999)). Briefly, six fractions were collected representing keratinocytes with a specific buoyant density and maturational state. By cytological examination, each fraction contains a distinct layer of keratinocytes with the small, basal layer keratinocytes with the highest density at the last fraction and the most superficial layer with highly differentiated squames with the lowest density in the first fraction.

Jagged-Related Peptides

Several different peptides were synthesized and utilized in these studies. A synthetic peptide (designated JAG-1) with Notch agonist activity in-vitro corresponding to Jagged-1 residues 188-204, (SEQ ID NO:11) is part of the DSL region, and is highly conserved between human Jagged-1 and Jagged-2. Other peptides included a second peptide with agonist activity designated R-JAG (SEQ ID NO:12) derived from the same region of Jagged-1, but with specific amino acid changes and a scrambled control peptide without agonist activity designated SC-JAG (SEQ ID NO:13).

Retroviral Vectors and Transduction of Normal Keratinocytes

The dominant negative IκBα cDNA (IκBαDN) was subcloned into the BamHI and NotI of LZRS and MGF-based retroviral expression vector as previously described (Qin et al., supra). The LZRS vector containing enhanced green fluorescent protein is described in the literature (Qin et al., supra). The Phoenix-Ampho retroviral packaging cells were obtained from American Type Culture Collection (Manassas, Va.). The packaging cells were cultured in Dulbecco's modified Eagle's medium and transfected with LZRS-IκBαDN vector by using CaCl₂ and 2× Hanks Balanced Salt Solution. After overnight incubation, the cells were fed with fresh medium and incubated at 32° C. for and additional 24-48 hours. The supernatants were collected for cell infection. The normal keratinocytes were seeded into 6-well plates and infected with 300 μl of viral supernatant in the presence of 4 μg/ml hexadimethrine bromide for 1 hour at 32° C., then the supernatant was removed and replaced with fresh medium, incubated at 37° C. in 5% CO₂ overnight.

NF-κB Luciferase Assay

To perform the luciferase-based NF-κB assays, normal keratinocytes were seeded in 6-well plates as previously described (Qin et al., supra). At approximately 50% to 60% confluence, the cells were co-transfected with 0.8 μg of pNF-κB-LUC vector (CLONTECH Laboratories, Palo Alto, Calif.) containing a firefly luciferase gene with K enhancer and 0.2 μg of PRL-TK plasmid DNA which contains Renella Luciferase gene to normalize the transfection efficiency. The same amount of control vector in which the κ enhancer was removed from pNF-κB-LUC was also used to co-transfect the cells.

DNA was transfected into keratinocytes using Fu-Gene 6 Transfection Reagent (Roche Molecular Biochemicals) according to the manufacturer's protocol. After 36 hours of transfection, cells were treated with either TNF-α (10³ U/ml; R&D Systems, Minneapolis, Minn.) or the indicated peptides.

The preparation of cell lysate and luciferase activity measurements were made with DUAL LUCIFERASE™ Reporter Assay System (Promega, Madison Wis.) according to the manufacturer's instructions. The sample was placed in a TD-20/20 luminometer (CLONTECH Laboratories) for detection of light intensity.

Antibodies

The anti-Notch 1, 3, 4 receptor antibodies, Notch 1 (SC-6014), Notch 3 (SC-7424), Notch 4 (SC-8644) antibodies and anti-Jagged-1 (SC-6011) antibody, and the anti-p50 (SC-7178) anti-p65 (SC-109), and anti IκBa (SC-371) were purchased from Santa Cruz Biotechnology Inc (Santa Cruz, Calif.). The anti-loricrin antibody was obtained from (BabCO) (PRB-145P; Richmond, Calif.). The anti-PKC-alpha and anti-keratin 1 antibodies were purchased from Upstate Biotechnologies Inc (Lake Placid, N.Y.).

IKKα Immunocomplex Kinase Assay

For immunoprecipitation 2 μg of anti-IKKα and anti-IKKβ were added to the whole cell lysate for 2 hours on ice. 30 μl of protein A-G sepharose beads were added and incubated at 40° C. for 90 minutes. Beads were washed 3 times in wash buffer, and the beads were boiled with 2xSDS sample buffer. Kinase activity was performed using GST-IκBα as substrate (Hu et al., Science, 284, 316-20 (1999)).

Electromobility Shift Assay (EMSA)

EMSA were done as described previously (Qin et al., supra). 5 μg of nuclear proteins were incubated on ice with 1 μg of poly(dI-dC) (Amersham Pharmacia Biotech) in a buffer containing 10 mM HEPES, 60 mM KCl, 1 mM dithiothreitol, 1 mM EDTA, and 4% Ficoll for 10 min. The ³²P-labeled double strand NF-κB binding oligonucleotide (10⁵ cpm) was then added to the reaction mixture for an additional 20 min on ice. The NF-κB oligonucleotide had the following sequence: SEQ ID NO:14 (5′-AGTTGAGGGGACTTTCCCAGGC-3′). For experiments involving supershift analysis, 2-4 μg of polyclonal antibodies against different subunits of NF-κB (Santa Cruz Biotech) were incubated with nuclear proteins for 30 min on ice prior to addition of ³²P-labeled NF-κB probe. All the reaction mixtures were resolved on 4% polyacrylamide gels.

Western Blot Analysis

Whole cell lysates, or nuclear and cytoplasmic extracts, were prepared to detect different proteins as previously described (Chaturvedi et al., J. Biol. Chem., 274, 2358-67 (1999)). Briefly, keratinocytes were washed with ice cold phosphate buffered saline (PBS), and harvested by scraping the monolayers into 1 ml PBS, and pelleted in a 1-5 ml microcentrifuge tube. The cell pellet was suspended in 400 μl of buffer A (10 mM HEPES, 10 mM KCl, 1 mM EDTA, 1 mM EGTA, 1 mM dithiothreitol, 0.5 mM phenylmethlysulfonyl fluoride, and 5 μg/ml leupeptin). After 20 min incubation on ice, 25 μl of 10% Nonidet P-40 was added and then centrifuged briefly. The supernatant represented the cytoplasmic extracts. The nuclear pellet was resuspended in 60-80 μl of buffer C (20 mM HEPES, 0.4 M NaCl, 1 mM EDTA, 1 mM EGTA, 1 mM dithiothreitol, 0.5 mM phenylmethylsulfonyl fluoride, and 5 μg/ml leupeptin) and incubated at 4° C. with shaking for 15 min. The nuclear debris was removed by centrifugation at 4° C.

For the whole cell lysate, cells were washed with cold PBS and incubated in CHAPS buffer on ice for 15 min. After centrifugation at 4° C., the supernatants were saved as whole cell lysate. The protein concentration of each sample was determined with Bio-Rad protein assay reagent. 30 μg of protein were loaded on 8-12.5% SDS-polyacrylamide gel, transferred to Immobilon-P (polyvinylidene difluoride) membrane and blocked in 5% powdered milk in TBST (50 mM Tris, pH 7.5, 150 mM NaCl, 0.01% Tween 20). The membrane was incubated with primary antibody overnight at 4° C. in 2.5% powdered milk in TBST and then was washed extensively with TBST, and incubated with 1:1500 anti-rabbit or mouse-HRP (Amersham Pharmacia Biotech). Proteins were visualized with the ECL detection kit (Amersham Pharmacia Biotech). The equivalent loading of proteins in each well was confirmed by Ponceau staining.

Living Epidermal Equivalent Systems

Reconstituted epidermis, known as epidermal equivalents were purchased from MatTek Corp., Ashland, Mass. These epidermal equivalents consist of several layers of relatively undifferentiated keratinocytes submerged in standard medium (DMEM plus EGF, insulin, hydrocortisone-free) as previously described (Kubilus et al., In vivo Toxicol., 9, 157-66 (1996)). In this system, exposure of the epidermal equivalent to an air-liquid interface is a potent stimulus for differentiation with creation of a stratum corneum that is comparable to normal human skin in vivo.

Histological Analysis and Immunohistochemistry

Portions of the epidermal equivalents were fixed in 10% neutral buffered formalin, embedded in paraffin and routinely stained with 1% hematoxylin and eosin. For immunohistochemical analysis, some sections were subjected to antigen retrieval using a standard microwave procedure (to detect Notch 1, 3, 4, Jagged-1, protein kinase C-alpha), while other staining (to detect loricrin) did not require antigen retrieval. When using Lab Tek cultures, these slides were rinsed in PBS and fixed with ice-chilled acetone for 10 mins prior to addition of primary antibodies. Cryostat sections of skin also were fixed using ice-cold acetone. In all cases, a highly sensitive avidin biotin peroxidase procedure (Vectastain Kit, Vector Labs, Burlingame, Calif.) was used with a positive red reaction product obtained using 3-amino-4-ethylcarbazole as the chromagen.

### Example 1

This Example Demonstrates Localization and Characterization of Notch Ligands and Receptors in Normal Human Skin

Immunostaining cryostat sections of normal human skin with a panel of antibodies directed against various members of the Notch receptor/ligand family revealed consistent results for all skin specimens examined. Immunostaining of Jagged-1 showed strong and diffuse predominantly plasma membrane staining of keratinocytes beginning in the supra-basal cell layer, involving multiple cell layers up to the granular cell layer producing a chicken-wire appearance. There was only rare to absent Jagged-1 staining of either the granular cell layer or the stratum corneum layer. There was only weak cytoplasmic expression of Jagged-1, and no significant nuclear staining. Notch 1 immunoreactivity was primarily confined to keratinocytes in the lower and mid level layers of the epidermis, with only faint and focal staining of the granular cell layer and the stratum corneum. Notch 1 was detected in these keratinocytes with a more stippled staining pattern of the plasma membrane, and while there was minimal cytoplasmic staining, occasional positive nuclear staining was observed. There was no immunoreactivity for Notch 2 in the epidermis using this antibody.

Immunostaining to detect Notch 3 revealed primarily localization to keratinocytes in the mid-layers and upper layers of the epidermis. Plasma membrane staining was more prevalent than cytoplasmic staining, and no consistent nuclear patterns were observed. In contrast to Jagged-1 and Notch 1 staining, the Notch 3 staining included more diffuse and consistent plasma membrane reactivity by keratinocytes near the granular cell layer. In addition, Langerhans cells were also positive for Notch 3. Notch 4 localization was primarily present in the suprabasal layers and included plasma membrane staining of keratinocytes near the granular cell layer. The Notch 4 staining also included peri-nuclear staining of the cytoplasm, and occasional nuclear staining, as well as Langerhans cells. Besides these epidermal-based staining profiles for Notch ligand/receptors, the adnexal structures such as hair follicles, sweat glands, and the underlying dermis, including dendritic cells and endothelial cells lining blood vessels were examined. Table I summarizes these immunolocalization results for the Notch ligand/receptors.

To confirm and extend these immunohistochemical-based studies, each cell layer of the epidermis was characterized by Western blot analysis. The six different bands from the Percoll gradient were initially analyzed for loricrin levels. Intact human skin contains a band of loricrin positive cells in the outermost layers of epidermis representing terminally differentiated keratinocytes. The presence of loricrin in the most buoyant bands of the Percoll gradient that represent keratinocytes from the granular cell layer and the stratum corneum, whereas keratinocytes from the mid-level stratum spinosum and basal cell layers were devoid of loricrin. Conversely, the least buoyant fraction derived from basal layer keratinocytes was observed to be devoid of keratin 1. When the identical protein fractions were analyzed for their Jagged-1 content, Jagged-1 was detectable primarily in the mid-epidermal layers, in good agreement with the immunostaining. Notch 1 was detectable in its mature, transmembrane form (120 kd), and was particularly abundant in the lower layers of the skin. Indeed, differential expression of Loricrin, keratin 1, Jagged-1, Notch and β-actin by keratinocytes was observed within the various cell layers. Thus, using immunostaining and immunoblot analysis of keratinocytes fractionated by maturational state, it appeared that Jagged-1 was strategically located to influence both early stages as well as late stages of keratinocyte differentiation.

### Example 2

This example demonstrates the ability of a source of a Notch agonist to activate NF-κB signaling in epithelial tissue.

Keratinocytes were transfected to allow use of luciferase-based NF-κB assay, and the activity of NF-κB determined using keratinocytes maintained in either a low calcium (0.07 mM) or high calcium (2 mM) medium. It was observed that exposure of keratinocytes for 18 hours to elevated calcium ion concentrations did not, by itself, induce NF-κB activity. Addition of JAG-1 peptide at the indicated concentrations did trigger NF-κB activation in high calcium, but not low calcium conditions. Treatment of keratinocytes with TNF-α served as a positive control for NF-κB activation under both low and high calcium conditions. To verify a link between JAG-1 activation, a retroviral vector with a dominant negative mutant (i.e. IκBα) for NF-κB activity was utilized. Compared to keratinocytes infected with an empty retroviral construct (i.e. Linker-control), the co-incubation with the IκBα retrovirus demonstrated that blocking NF-κB activity inhibited the ability of either TNF-α or JAG-1 to activate NF-κB.

Activation of NF-κB in keratinocytes can be mediated by p50 and/or p65 subunits that become liberated from their cytoplasmic location and rapidly translocated to the nucleus. To assess the molecular composition of the NF-κB dimers mediating the luciferase-based activation signals, nuclei from cultured keratinocytes were isolated before and after JAG-1 exposure followed by Western blot analysis. 18 hours following an increase in calcium ion concentration to 2 mM, only a low intranuclear amount of p50 was detected, accompanied by a barely detectable level of p65. However, addition of JAG-1 peptide induced markedly enhanced intranuclear levels for both p50 and p65 subunits at 30 minutes and 1 hour. The rapid induction of these heterodimers was followed by a subsequent decline in their levels at 2 hours, 6 hours, and 24 hours; being greater for the p50 subunit compared to the p⁶⁵ subunit.

To confirm and extend the luciferase-based assay of NF-κB activity, and the intranuclear immunoblot analysis, nuclear extracts were prepared and incubated with a specific probe containing NF-κB binding sites. These results indicated that JAG-1 peptide can activate NF-κB transcriptional activity, which includes the heterodimers p50 and p65, as well as the p50/p50 homodimers.

Since proteins potentially responsible for sequestering p50/p65 subunits in the cytoplasm include IκB proteins, cytoplasmic extracts from the same cultures just mentioned using nuclear extracts were examined by Western blot analysis. The most prominent inhibitory protein was IκBα in keratinocytes before exposure to JAG-1 peptide. After addition of JAG-1 peptide, no consistent decline was observed after 24 hours. The relatively low constitutive level of IκBα was slightly reduced at the 7-minute to 6-hour time points, and became undetectable after 24 hours.

Notch-1 itself has been previously identified as being capable of binding NF-κB (Guan et al., J. Exp. Med., 183, 2025-32 (1996)). There was no significant change in the levels of the 120 Kd form of Notch 1 during the initial 2 hours of JAG-1 peptide exposure, but 6 hours and 24 hours after JAG-1 addition, Notch 1 levels decreased to barely detectable levels. To further explore the cellular localization of Jagged-1 and Notch receptors, before and after exposure to JAG-1 peptide, monolayers of cultured keratinocytes were immunostained. Keratinocytes maintained in a 2 mM calcium ion containing medium displayed prominent Jagged-1 on their plasma membranes producing a “chicken-wire” appearance, resembling the staining pattern for suprabasal layer keratinocytes in intact skin. Twenty-four hours after addition of JAG-1 peptide, there was loss of this plasma membrane staining for Jagged-1, with some peri-nuclear cytoplasmic re-distribution. Notch-1 was also predominantly localized to the plasma membrane in the untreated cultures resembling the keratinocytes in the mid-layer of the epidermis, in normal skin, and was re-distributed to the cytoplasm with occasional nuclear positivity 24 hours after JAG-1 peptide stimulation confirming the Western blot analysis.

Notch 3 immunolocalization revealed both constitutive expression at the plasma membrane as well as diffuse cytoplasmic staining, with disappearance of both membrane and cytosolic Notch 3 after addition of JAG-1 peptide. Only weak and focal staining was observed for the plasma membrane, cytoplasm and nuclei for Notch 4 in keratinocyte cultures before addition of JAG-1 peptide, with loss of membrane and cytosolic Notch 4, after JAG-1 peptide stimulation but retention of focal nuclear staining. These data indicate that JAG-1 treatment can induce cellular redistribution of Notch 1, Notch 3, and Notch 4, suggesting that this ligand causes simultaneous activation of all these Notch receptors in keratinocytes.

### Example 3

This example demonstrates the ability of a source of a Notch agonist to activate the kinase activity of IKKα in epithelial tissue.

The protein kinase complex that phasphorylates IκBs contains two catalytic subunits (IKKα and IKKβ). Since IKKα appears to be involved in terminal differentiation and corneogenesis of the epidermis, the ability of JAG-1 to activate the kinase activity of IKKα was investigated. To measure activation kinetics, keratinocyte extracts before and after various time points following addition of JAG-1 or SC-JAG (control) were immunoprecipitated with an anti IKKα antibody and incubated with a glutathione S-transferase—IκBα (1-54) as a substrate. While TNF-α or the control peptide had little effect on the activation of IKKα, addition of either JAG-1 or R-JAG did activate the kinase activity of IKKα after 30 and 60 minutes. This increase in kinase activity was not accompanied by any significant change in the amount of IKKα present immediately following JAG-1 stimulation.

### Example 4

This example demonstrates that the induction of terminal differentiation/cornification increases Notch ligand/receptors

While addition of JAG-1 peptide was shown to influence the levels and cellular distribution profiles for Jagged-1 itself, as well as Notch 1, 3, 4 receptors, the relationship between induction of differentiation and the Notch pathway was explored. For these experiments a living epidermal equivalent model system was used, because it provided an opportunity to examine the individual components of the Notch pathway (i.e. Notch ligand and receptors) before, and after, induction of differentiation for keratinocytes maintained in a multi-layered or stratified configuration that closely resembles normal human skin. Epidermal equivalents consist of keratinocytes grown to produce a multi-layered structure that are initially prepared using submerged keratinocytes. Following this initial preparation, the cultures are exposed to an air-liquid interface in a moistened chamber for three days that can initiate the early stage of differentiation, designated as day 0. Under these conditions, approximately 5-7 layers are produced with a progressive flattening of the top level keratinocytes, which can include occasional cells with keratohyalin granules, and focal appearance of loricrin positive cells and corneocytes. When cultures arrived at day 0, they can either be re-submerged in medium or lifted-up and maintained on a support to create an air-liquid interface within a tissue culture well. After lifting of the cultures, the multi-layered keratinocytes are exposed to the air, with the subjacent membrane being moistened and fed only from the underlying medium within the well. Lifting the cultures at day 0, triggers the terminal differentiation process, which typically is completed within about 5 to about 7 days.

Examination of day 5 air-liquid interface cultures revealed a greater flattening and stratification of all keratinocyte layers, with appearance of a granular cell layer, diffuse loricrin positivity creating a continuous band-like effect at the top layer of viable keratinocytes, accompanied by a thick stratum corneum including stacked-layers of adherent and cohesive corneocytes. The final morphological appearance and loricrin expression pattern of these lifted epidermal equivalents was observed to be virtually undistinguishable from normal human skin.

To determine if Notch ligand/receptors were being modulated by the air-liquid interface-induced triggering of terminal differentiation, immunostaining of these epidermal equivalents was performed. Formalin-fixed paraffin embedded sections of the epidermal equivalents before being lifted revealed focal and faint plasma membrane staining to detect Jagged-1 in mid-level keratinocytes. Similarly, staining to detect Notch-1, Notch 3, and Notch 4 revealed only faint staining to a variable extent primarily confined to the plasma membrane of keratinocytes. However, five days after being lifted, cultures at the air-liquid interface revealed enhanced expression of Jagged-1, as well as the Notch receptors. The staining profiles in these lifted cultures included an increase predominantly in the cytoplasmic compartment with some plasma membrane staining, but no detectable nuclear localization. In addition to these immunolocalized studies, immunoblot assays were also performed using cell extracts derived from keratinocytes in epidermal equivalents before and after being lifted to an air-liquid interface. It was also observed that levels of Jagged-1 and Notch 1 were increased after the induction of terminal differentiation.

### Example 5

This example demonstrates the ability of a source of a Notch agonist to trigger terminal differentiation in cultured keratinocytes.

To determine if the JAG-1 peptide mediated activation of IKKα kinase activity and NF-κB signaling by cultured keratinocytes would result in terminal differentiation, monolayers were treated with JAG-1. In addition, these experiments were undertaken to determine if the elevated levels of Notch ligand and receptors observed when keratinocytes were undergoing differentiation, were actually responsible for the induction of differentiation. Unlike mouse keratinocytes in vitro that undergo terminal differentiation in response to elevated extracellular calcium ion concentration, human keratinocytes behave differently. Raising the extracellular calcium ion concentration to 2 mM does not induce stratification of the monolayer, or trigger significant levels of proteins involved in terminal differentiation—loricrin or involucrin. As early as 3 days following addition of JAG-1 peptide or R-JAG peptide to keratinocyte cultures maintained in 2 mM calcium, keratinocytes began piling-up to form 3-dimensional stratified clusters including the appearance of keratinocyte granules. Moreover, immunostaining demonstrated the strong induction of both loricrin and involucrin with a gradient of intensity in staining, beginning with mild to moderate staining of keratinocytes at the bottom layer, and increasing in intensity by keratinocytes that had moved-up to the top layers within those stratified clusters. These observations are consistent with a causative role of JAG-1 in inducing terminal differentiation.

### Example 6

This example demonstrates the ability of a source of a Notch agonist to trigger differentiation/corneogenesis and apoptosis resistance in epidermis.

The Living epidermal equivalent model system was employed to ascertain whether the JAG-1 peptide can mediate terminal differentiation/corneogenesis and apoptosis resistance. If the day 0 multi-layered keratinocyte cultures (see Example 4) were re-submerged in medium (i.e. not lifted), by day 5 the keratinocytes remained rather cuboidal (rather than becoming flattened), and underwent apoptosis. Scattered keratinocytes contained pyknotic nuclei and appeared shrunken with partial collapse of the epidermis unaccompanied by the increased appearance for either a granular cell layer, loricrin positivity, or cornification. However, when JAG-1 peptide was added to the submerged cultures at the same time as parallel cultures were lifted to an air-liquid interface, several notable differences were observed on day 5. First, addition of JAG-1 to the submerged cultures reduced the apoptotic process as viable keratinocytes become more flattened and prominent keratinocytes granules appear as seen earlier producing a granular cell layer. In addition there was diffuse loricrin expression by keratinocytes in the epidermal equivalent with accentuation of the keratinocytes at the top layer, and a thick stratum corneum layer with cohesive corneocytes. Comparing the submerged cultures treated with JAG-1 peptide to cultures exposed to an air-liquid interface reveals a remarkable similarity to normal human skin.

When a living epidermal equivalent model system was created using cultured keratinocytes that had been continuously submerged from the onset of the culture for 5 days (i.e., never exposed to a moistened air/liquid interface), addition on day 0 (i.e., upon arrival) of either JAG-1 (50 μM) or R-JAG (40 μM), but not the control SC-JAG (40 μM) triggered terminal differentiation and cornification 4 days later. These observations indicate that JAG-1 and R-JAG can trigger terminal differentiation and cornification, which in habiting premature apoptosis in epithelial tissue

### Example 7

This example demonstrates that a source of a Notch agonist Activates Protein Kinase C (PKC) activity in epithelial cells

Using keratinocyte monolayers, exposure to JAG-1 peptide induced translocation of PKC-alpha from the cytoplasm to the plasma membrane, which is indicative of PKC activation. In addition, examination of the epidermal equivalents exposed to JAG-1 revealed plasma membrane localization for PKC-alpha in the upper layers of differentiated keratinocytes. These results confirm a role for PKC activation mediated by JAG-1 in the differentiation process involving keratinocytes.

### Example 8

This example demonstrates that aggressive melanoma can be identified by assaying for expression of Notch signaling components.

Eight melanoma cell lines were selected for study, four of which (MUM-2B, A375P-VAR, C918, and C8161) are phenotypically aggressive-type lymphoma cell lines, while four others (MUM-2C, A375P, OCHIA, and C81-61) are non-aggressive. These cell lines were plated on separate 8-well Lab Tek slides and allowed to grow until confluence. At confluence, the cultures were fixed and stained using well-characterized antibodies specific to one of either three Notch ligands (JAG-1, JAG-2, and Delta), three Notch receptors (Notch-1, Notch-2, or Notch-4), or one of three endothelial cell markers/adhesion molecules (CD31, CD34, or CD54). The labeled cells then were stained with a secondary antibody and the staining patterns examined. It was observed that all of the aggressive melanoma cell lines stained heavily for each of the Notch ligands and receptors (consistent with over expression of the proteins), while the non-aggressive cell lies stained much more modestly for such proteins. Also, it was observed that the aggressive melanoma cell lines stained somewhat more heavily for the three cell adhesion molecules. These results reveal that assaying for Notch ligand and Notch receptor expression in a melanoma cell culture can serve as a molecular marker, and thus a basis for diagnosing, aggressive melanoma.

### Example 9

This example demonstrates that inhibiting the Notch pathway can be retard the progression of aggressive melanoma.

Two cultures of MOM-2b cells (an aggressive melanoma cell line) were cultured in standard medium alone or in the presence of 100 nM of a gamma secretase inhibitor, which interferes with Notch signaling (Supont Compound E; Seiffaert et al., J. Biol. Chem., 275, 34086 (2000)). The cells then were lysed and assayed for the presence of Notch-1 by Western blotting. It was observed that, at this concentration, compound E completely blocked the activation of Notch-1.

In a second experiment, MOM-2b cells were pretreated overnight with 100 nM compound E or untreated culture medium and then analyzed for invasive behavior by culturing them on MATRIOEL™ coated filters, into which aggressive melanoma will invade. After 20 hours, cells that have not invaded through the filters were discarded and cells that had migrated into the top of the filters were stained, lysed, and then assayed for absorbance at 350 nM to quantitate the number of cells that had invaded through the filter. It was observed that the culture pretreated with compound E had an OD₃₅₀ of 0.378, while the control culture exhibited an OD₃₅₀ of 1.484. These results indicate that pretreatment with Compound E (and thus inhibition of Notch-1 production) inhibited aggressive behavior in melanoma cells by 75%. In other experiments, it was also observed that compound E did not exhibit cytotoxicity at concentrations <300 nM.

### Example 10

This example demonstrates that cutaneous T-cell lymphoma can be identified by assaying for expression of Notch signaling components

Skin lesions from several patients with CTCL were stained substantially as described in Example 8. Upon histological examination, it was apparent that the keratinocytes within the lesions overexpressed JAG-1, JAG-2, and Delta, whereas the malignant intra-epidermal T cells overexpressed Notch-3 and Notch-4. This result is consistent with prior observations that NFκB activation is associated with CTCL, as Notch signaling can stimulate NFκB expression. These results reveal that assaying for Notch receptor expression in a lesion can serve as a molecular marker for CTCL. Furthermore, antagonizing Notch signal (or the “Notch pathway”), which blocks or attenuates NFκB expression, can induce apoptosis in malignant lymphoma cells.

### Incorporation by Reference

All sources (e.g., inventor's certificates, patent applications, patents, printed publications, repository accessions or records, utility models, world-wide web pages, and the like) referred to or cited anywhere in this document or in any drawing, Sequence Listing, or Statement filed concurrently herewith are hereby incorporated into and made part of this specification by such reference thereto.

### Guide to Interpretation

The foregoing is an integrated description of the invention as a whole, not merely of any particular element of facet thereof. The description describes “preferred embodiments” of this invention, including the best mode known to the inventors for carrying it out. Of course, upon reading the foregoing description, variations of those preferred embodiments will become obvious to those of ordinary skill in the art. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

As used in the foregoing description and in the following claims, singular indicators (e.g., “a” or “one”) include the plural, unless otherwise indicated. Recitation of a range of discontinuous values is intended to serve as a shorthand method of referring individually to each separate value falling within the range, and each separate value is incorporated into the specification as if it were individually listed. As regards the claims in particular, the term “consisting essentially of” indicates that unlisted ingredients or steps that do not materially affect the basic and novel properties of the invention can be employed in addition to the specifically recited ingredients or steps. In contrast, the terms “comprising” or “having” indicate that any ingredients or steps can be present in addition to those recited. The term “consisting of” indicates that only the recited ingredients or steps are present, but does not foreclose the possibility that equivalents of the ingredients or steps can substitute for those specifically recited.

What is claimed is:

1. A method of inducing differentiation of at least one epithelial cell comprising exogenously providing at least one source of at least one Notch agonist to at least one epithelial cell whereby the Notch pathway is activated within at least one epithelial cell such that differentiation of at least one epithelial cell is induced.

2. The method of claim 1, wherein at least one epithelial cell is a keratinocyte.

3. The method of claim 1, wherein at least one epithelial cell is a pre-malignant cell.

4. The method of claim 1, wherein at least one source of at least one Notch agonist is at least one constitutively active derivative of the Notch protein which is provided to the at least one epithelial cell intracellularly.

5. The method of claim 1, wherein at least one source of at least one Notch agonist is at least one antisense nucleotide targeting at least one endogenous inhibitor(s) or inhibiting pathway(s) that antagonizes and/or down-regulate(s) the Notch pathway, which is provided to the at least one epithelial cell intracellularly to attenuate the inhibition, and thereby activate, the Notch pathway within the at least one epithelial cell.

6. The method of claim 1, wherein at least one source of at least one Notch agonist is at least one ligand for at least one Notch protein, which is provided extracellularly to at least one epithelial cell.

7. The method of claim 6, wherein at least one Notch ligand is selected from the group of ligands consisting of Jagged 1, Jagged 2, Lunatic-Fringe, Manic-Fringe, Radical Fringe, Delta, Serrate, and active fragments and derivatives thereof.

8. The method of claim 6, wherein at least one Notch ligand is a protein having a sequence of amino acids consisting essentially of one of SEQ ID NOs: 2-12, active fragments, and derivatives thereof.

9. The method of claim 6, wherein at least one ligand is a Jagged 1 derivative derived from the Delta/Serrate/LAG-2 domain of hJagged 1.

10. The method of claim 6, wherein at least one ligand is provided to at least one epithelial cell in a composition comprising the Notch ligand and a physiologically-compatible carrier.

11. The method of claim 6, wherein at least one ligand is provided by inducing at least one second cell in the region of the epithelial cell to produce the ligand.

12. The method of claim 11, wherein at least one second cell is induced to produce the ligand by providing at least one chemical agent to the second cell to which the second cell responds by producing at least one Notch ligand.

13. The method of claim 11, wherein at least one expression cassette encoding at least one Notch ligand is introduced into at least one second cell such that the second cell produces at least one Notch ligand.

14. The method of claim 1, wherein at least one epithelial cell is in vivo.

15. The method of claim 1, wherein at least one epithelial cell is ex vivo.

16. The method of claim 1, wherein at least one epithelial cell is within cutaneous epithelial tissue or epidermal equivalent.

17. The method of claim 1, wherein at least one epithelial cell is within extracutaneous epithelium selected from the group of epithelial tissues consisting of oral mucosal, cornea, gastrointestinal epithelia, urogenital epithelia, and respiratory epithelia.

18. A method for inducing formation of at least one barrier within epithelium comprising exogenously providing at least one source of at least one Notch agonist to at least one epithelial cell within the epithelium such that at least one epithelial cell is induced to form a barrier within the epithelium.

19. The method of claim 18, wherein the barrier is a stratum corneum.

20. The method of claim 18, wherein at least one source of at least one Notch agonist is at least one constitutively active derivative of the Notch protein which is provided to the at least one epithelial cell intracellularly.

21. The method of claim 18, wherein at least one source of at least one Notch agonist is at least one antisense nucleotide targeting at least one endogenous inhibitor(s) or inhibiting pathway(s) that antagonizes and/or down-regulate(s) the Notch pathway, which is provided to the at least one epithelial cell intracellularly to attenuate the inhibition, and thereby activate, the Notch pathway within the at least one epithelial cell.

22. The method of claim 18, wherein at least one source of at least one Notch agonist is at least one ligand for at least one Notch protein, which is provided extracellularly to at least one epithelial cell.

23. The method of claim 22, wherein at least one Notch ligand is selected from the group of ligands consisting of Jagged 1, Jagged 2, Lunatic-Fringe, Manic-Fringe, Radical Fringe, Delta, Serrate, and active fragments and derivatives thereof.

24. The method of claim 22, wherein at least one Notch ligand is a protein having a sequence of amino acids consisting essentially of one of SEQ ID NOs: 2-12, active fragments, and derivatives thereof.

25. The method of claim 22, wherein at least one ligand is a Jagged 1 derivative derived from the Delta/Serrate/LAG-2 domain of hJagged 1.

26. The method of claim 22, wherein at least one ligand is provided to at least one epithelial cell in a composition comprising the Notch ligand and a physiologically-compatible carrier.

27. The method of claim 22, wherein at least one ligand is provided by inducing at least one second cell in the region of the epithelial cell to produce the ligand.

28. The method of claim 27, wherein at least one second cell is induced to produce the ligand by providing at least one chemical agent to the second cell to which the second cell responds by producing at least one Notch ligand.

29. The method of claim 27, wherein at least one expression cassette encoding at least one Notch ligand is introduced into at least one second cell such that the second cell produces at least one Notch ligand.

30. The method of claim 18, wherein at least one epithelial cell is in vivo.

31. The method of claim 18, wherein at least one epithelial cell is ex vivo.

32. The method of claim 18, wherein at least one epithelial cell is within cutaneous epithelial tissue or epidermal equivalent.

33. The method of claim 18, wherein at least one epithelial cell is within extracutaneous epithelium selected from the group of epithelial tissues consisting of oral mucosal, cornea, gastrointestinal epithelia, urogenital epithelia, and respiratory epithelia.

34. A method for producing differentiated epidermis comprising culturing undifferentiated epidermal tissue in the presence of at least one source of a Notch agonist to the epidermal tissue whereby the Notch Pathway is activated within at least one cell of the epidermal tissue such that differentiation of the epidermis is induced.

35. The method of claim 34, wherein the epidermal tissue is cultured by being submerged in a solution comprising at least one source of a Notch agonist.

36. A method of assaying for genetic propensity of a patient to develop a disorder associated with epithelial barrier formation, the method comprising obtaining DNA or RNA from the patient, determining a characteristic of the DNA or RNA from the patient that encodes at least one protein selected from Notch proteins and Notch ligands, and assessing whether characteristic differs from the corresponding wild-type characteristic.

37. The method of claim 36, wherein genomic DNA is obtained and sequenced.

38. The method of claim 36, wherein cDNA is obtained and sequenced.

39. The method of claim 36, wherein RNA is obtained and sequenced.

40. A protein having a sequence of amino acids consisting essentially of the amino acid sequence of one of SEQ ID NOs:9-12 or a conservative substituent thereof.

41. The protein of claim 40, wherein the protein is a Notch ligand.

42. A method of retarding the progression of a pre-malignant epithelial cell towards malignancy, comprising exogenously providing at least one source of at least one Notch agonist to a pre-malignant epithelial cell, whereby the progression of the cell towards malignancy is retarded.

43. A method of retarding the progression of a skin cancer, comprising administering to skin cancerous cells an agonist or antagonist of the Notch pathway, whereby upon contact with the agonist or antagonist of the Notch pathway, the progression of the skin cancer is retarded.

44. The method of claim 43, wherein the skin cancer is selected from the group of skin cancers consisting of aggressive melanoma, aggressive cutaneous T-cell Lymphoma (CTCL), aggressive squamous cell carcinoma, and aggressive basal cell carcinoma and the method involves administering an antagonist of the Notch pathway.

45. The method of claim 43, wherein the cancer is aggressive melanoma.

46. The method of claim 43, wherein the cancer presents as aggressive CTCL that presents in the skin.

47. The method of claim 43, wherein the antagonist is a gamma secretase inhibitor.

48. The method of claim 43, wherein the antagonist is a protein having a sequence of amino acids consisting essentially of the amino acid sequence of one of SEQ ID NOs:16 or 18 or a conservative substituent thereof.

49. The method of claim 43, wherein the skin cancer is selected from the group of skin cancers consisting of non-agressive melanoma, non-aggressive cutaneous T-cell Lymphoma (CTCL), non-aggressive squamous cell carcinoma, and non-aggressive basal cell carcinoma and the method involves administering an agonist of the Notch pathway.

50. A method of diagnosing aggressive melanoma, comprising obtaining a tissue biopsy from a patient, assaying the tissue biopsy for the overexpression of a protein selected from the group of proteins consisting of Notch ligands, Notch receptors, or endothelial cell markers/adhesion molecules, whereby the overexpression of one or more of the proteins leads to the positive diagnosis of aggressive melanoma within the patient.

51. The method of claim 50, wherein the tissue biopsy is assayed for one or more proteins from the group of proteins consisting of JAG-1, JAG-2, and Delta, Notch-1, Notch-2, Notch-3, Notch-4, CD31, CD34, or CD54.

52. The method of claim 51, wherein the tissue biopsy is assayed for the overexpression of two or more of the proteins, and the overexpression of two or more of the proteins leads to the positive diagnosis of aggressive melanoma within the patient

53. A method of diagnosing CTCL, comprising obtaining a tissue biopsy from a patient, assaying the tissue biopsy for the expression of a T-cell-specific marker and a Notch receptor in a first cell within the tissue biopsy, and for the expression of a Notch ligand in a second cell within the tissue sample, whereby the staining pattern leads to the positive diagnosis of aggressive lymphoma within the patient


SEQUENCE LISTING (18 sequences)

SEQ ID NO: 1 - DNA, 7332 bp, Artificial Sequence
  Constitutively Active Notch-1
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaacgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccaggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgcccgtgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttttactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatgtgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgcctacggggcacctgccaggacccggacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacagcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaacgagtgtgaatccaacccttgtgtcaacggcggcacctgcaaagacatgaccagtggcatcgtgtgcacctgccgggagggcttcagcggtcccaactgccagaccaacatcaacgagtgtgcgtccaacccatgtctgaacaagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacggctggggccaaagggcagacctgtgaggtcgacatcaacgagtgcgttctgagcccgtgccggcacggcgcatcctgccagaacacccacggcgsstaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgtagtcaatgagtgcgactcacgaccctgcctgctaggcggcacctgtcaggacggtcgcggtctccacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgatgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcngcagcatggccagctctggttccctgagggcttcaaagtgtctgaggccagcaagaagaagcggcgggagnccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggayggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtg

SEQ ID NO: 2 - protein, 2444 aa, Artificial Sequence
  misc_feature (891)..(891): The ′Xaa′ at location 891 stands for Gly, or Ala.
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPRLCRHGGTCHNEVGSYRCVCRATHTGPNCERPYVPCSPSPCQNGGTCRPTGDVTHECACLPGFTGQNCEENIDDCPGNNCKNGGACVDGVNTYNCPCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCMCMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRLRGTCQDPDNAYLCFCLKGTTGPNCEINLDDCASSPCDSGTCLDKIDGYECACEPGYTGSMCNSNIDECAGNPCHNGGTCEDGINGFTCRCPEGYHDPTCLSEVNECNSNPCVHGACRDSLNGYKCDCDPGWSGTNCDINNNECESNPCVNGGTCKDMTSGIVCTCREGFSGPNCQTNINECASNPCLNKGTCIDDVAGYKCNCLLPYTGATCEVVLAPCAPSPCRNGGECRQSEDYESFSCVCPTAGAKGQTCEVDINECVLSPCRHGASCQNTHGXYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHVVNECDSRPCLLGGTCQDGRGLHRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRXQHGQLWFPEGFKVSEASKKKRREXLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADV

SEQ ID NO: 3 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 4 - protein, 1238 aa, Homo sapiens [sequence omitted]

SEQ ID NO: 5 - protein, 257 aa, Homo sapiens
PLAEPLAPRDVFIAVKTTKKFHRARLDLLLETWISRHKEMTFIFTDGEDEALARHTGNVVITNCSAAHSRQALSCKMAVEYDRFIESGRKWFCHVDDDNYVNLRALLLLLASYPHTLDVYVGKPSLDRPIQAMERVSENKVRPVHFWFATGGAGFCISRGLALKMSPWASGGHFMNTAERIRLPDDCTIGYIVEALLGVPLIRSGLFHSHLENLQQVPTSELHEQVTLSYGMFENKRNAVHVKGPFSVEADPSRWGN

SEQ ID NO: 6 - protein, 321 aa, Homo sapiens [sequence omitted]

SEQ ID NO: 7 - protein, 191 aa, Homo sapiens
MSRARGALCRACLALAAALAALLLLPLPLPRAPAPARTPAPAPRAPPSRPAAPSLRPDDVFIAVKTTRKNHGPRLRLLLRTWISRARQQTFIFTDGDDPELELQGGDRVINTNCSAVRTRQALCCKMSVEYDKFIESGRKWFCHVDDDNYVNARSLLHLLSSFSPSQDVYLGRPSLDHPIEATERVQGGRT

SEQ ID NO: 8 - protein, 1404 aa, Drosophila melanogaster
MFRKHFRRKPATSSSLESTIESADSLGMSKKTATKRQRPRHRVPKIATLPSTIRDCRSLKSACNLIALILILLVHKISAAGNFELEILEISNTNSHLLNGYCCGMPAELRATKTIGCSPCTTAFRLCLKEYQTTEQGASISTGCSFGNATTKILGGSSFVLSDPGVGAIVLPFTFRWTKSFTLILQALDMYNTSYPDAERLIEETSYSGVILPSPEWKTLDHIGRNARITYRVRVQCAVTYYNTTCTTFCRPRDDQFGHYACGSEGQKLCLNGWQGVNCEEAICKAGCDPVHGKCDRPGECECRPGWRGPLCNECMVYPGCKHGSCNGSAWKCVCDTNWGGILCDQDLNFCGTHEPCKHGGTCENTAPDKYRCTCAEGLSGEQCEIVEHPCATRPCRNGGTCTLKTSNRTQAQVYRTSHGRSNMGRPVRRSSSMRSLDHLRPEGQALNGSSSSGLVSLGSLQLQQQLAPDFTCDCAAGWTGPTCEINIDECAGGPCEHGGTCIDLIGGFRCECPPEWHGDVCQVDVNECEAPHSAGIAANALLTTTATAIIGSNLSSTALLAALTSAVASTSLAIGPCINAKECRNQPGSFACICKEGWGGVTCAENLDDCVGQCRNGATCIDLVNDYRCACASGFTGRDCETDIDECATSPCRNGGECVDMVGKFNCICPLGYSGSLCEEAKENCTPSPCLEGHCLNTPEGYYCHCPPDRAGKHCEQLRPLCSQPPCNEGCFANVSLATSATTTTTTTTTATTTRKMAKPSGLPCSGHGSCEMSDVGTFCKCHVGHTGTFCEHNLNECSPNPCRNGGICLDGDGDFTCECMSGWTGKRCSERATGCYAGQCQNGGTCMPGAPDKALQPHCRCAPGWTGLFCAEAIDQCRGQPCHNGGTCESGAGWFRCVCAQGFSGPDCRINVNECSPQPCQGGATCIDGIGGYSCICPPGRHGLRCEILLSDPKSACQNASNTISPYTALNRSQNWLDIALTGRTEDDENCNACVCENGTSRCTNLWCGLPNCYKVDPLSKSSNLSGVCKQHEVCVPALSETCLSSPCNVRGDCRALEPSRRVAPPRLPAKSSCWPNQAVVNENCARLTILLALERVGKGASVEGLCSLVRVLLAAQLIKKPASTFGQDPGMLMVLCDLKTGTNDTVELTVSSSKLNDPQLPVAVGLLGELLSSRQLNGIQRRKELELQHAKLAALTSIVEVKLETARVADGSGHSLLIGVLCGVFIVLVGFSVFISLYWKQRLAYRTSSGMNLTPSLDALRHEEEKSNNLQNEENLRRYTNPLKGSTSSLRAATGMELSLNPAPELAASAASSSALHRSQPLFPPCDFERELDSSTGLKQAHKRSSQILLHKTQNSDMRKNTVGSLDSPRKDFGKRSINCKSMPPSSGDEGSDVLATTVMV

SEQ ID NO: 9 - protein, 17 aa, Artificial Sequence
  JAG-1b Notch ligand
ADDYYYGFGANKFGRPR

SEQ ID NO: 10 - protein, 21 aa, Artificial Sequence
  misc_feature: JAG-1c Notch ligand
ADDYYYGFGANKFGRPRDDFF

SEQ ID NO: 11 - protein, 17 aa, Artificial Sequence
  misc_feature: JAG-1 Notch ligand
CDDYYYGFGCNKFCRPR

SEQ ID NO: 12 - protein, 19 aa, Artificial Sequence
  misc_feature: r-JAG1 Notch ligand
CDDYYYGFGCNKFGRPRDD

SEQ ID NO: 13 - protein, 17 aa, Artificial Sequence
  misc_feature: Scrambled JAG peptide
RCGPDCFDNYGRYKYCF

SEQ ID NO: 14 - DNA, 22 bp, Artificial Sequence
  misc_feature: NF kappa B oligonucleotide
agttgaggggactttcccaggc

SEQ ID NO: 15 - DNA, 282 bp, Artificial Sequence
  misc_feature: Human Notch repeats 11-12 - a Notch antagnoist polypeptide
atggctgcagaattccatcatcatcatcatcatcaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatgtgcatgcccggctacgagggtgtgcactgcgaggtcaacacatgatga

SEQ ID NO: 16 - protein, 92 aa, Artificial Sequence
  Misc (1)..(92): Notch antagonist polypeptide containing tag sequence
MAAEFHHHHHHQDVDECSLGANPCEHAGKCINTLGSFECQCLQGYTGPRCEIDVNECVSNPCQNDATCLDQIGEFQCMCMPGYEGVHCEVNT

SEQ ID NO: 17 - DNA, 249 bp, Artificial Sequence
  exon (1)..(249): Encodes human Notch repeats 11-12
caggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatgtgcatgcccggctacgagggtgtgcactgcgaggtcaacacatgatga

SEQ ID NO: 18 - protein, 81 aa, Artificial Sequence
  misc (1)..(81): Human Notch repeats 11 and 12 - functions as Notch antagonist
QDVDECSLGANPCEHAGKCINTLGSFECQCLQGYTGPRCEIDVNECVSNPCQNDATCLDQIGEFQCMCMPGYEGVHCEVNT